(19) United States
(12) Patent Application Publication
(10) Pub. No.: US 2003/0165849 A1
(43) Pub. Date: Sep. 4, 2003

(54) Methods and reagents for introducing a sulfhydryl group into the 5'-terminus of RNA

(76) Inventors: Biliang Zhang, Shrewsbury, MA (US); Zhiyong Cui, Worcester, MA (US); Lei Zhang, Agawam, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 09/996,139

(22) Filed: Nov. 27, 2001

Related U.S. Application Data
(60) Provisional application No. 60/253,564, filed on Nov. 28, 2000.

(51) Int. Cl. C12Q 1/68; C07H 21/02; C12P 19/34

(52) U.S. Cl. 435/6; 435/91.2; 536/23.1

(57) ABSTRACT

Methods for synthesizing RNA molecules whose 5′-terminus comprises a thiol group are disclosed. The present invention discloses the formation of 5′-HS-PEG-GMP-RNA and 5′-GMPS-RNA which upon alkaline phosphatase treatment, independently lead to a 5′-HS-RNA molecule.

### RELATED APPLICATION

This application claims the benefit of United States Provisional Patent Application Serial No. 60/253,564, filed Nov. 28, 2000, entitled “Methods for Introducing a Sulfhydryl Group into the 5′-Terminus of RNA”. The teachings of the foregoing application is incorporated herein by reference.

### BACKGROUND OF THE INVENTION

RNA molecules play critical roles in many cellular processes and they are potential targets for drug discovery. The development of methods for studying the molecular details of the complex interactions and essential functions of RNA in cellular metabolism is challenging. Site-specific substitution and derivatization provide powerful tools for studying RNA structure and function. Although solid phase chemical synthesis can be used to introduce functional groups at any specific position, its use is limited to oligonucleotides shorter than approximately 40 nucleotides. Investigation of larger RNA molecules faces a limited number of methodologies for site-specific modification and substitution. Phosphorothioate modification is one of the most popular methods for functionalizing the 5′-terminus of RNA by a transcription or kinase reaction, but only a low labeling efficiency of terminal phosphorothioate with fluorophores has been reported and, importantly, fluorophores are the most attractive probes for RNA structure.

A sulfhydryl group is a special reactive group that can be incorporated into nucleic acids. The thiol-reactive functional groups are primarily alkylating reagents, including iodoacetamides, maleimides, benzylic halides, and bromomethylketones. The thiol group has a unique property that is its ability to undergo a thiol-disulfide exchange reaction. A pyridyl dithiol group is a popular type of thiol-disulfide exchange functional group used in the construction of cross-linkers or modification reagents. A pyridyl disulfide will readily undergo an interchange reaction with a free sulfhydryl to yield a single mixed disulfide product. Once a disulfide linkage is formed, it can be cleaved using disulfide reducing agents (e.g., dithiothreitol, “DTT”). However, the introduction of free thiol groups into the termini of RNA has not been reported.

Modifications of the 5′-terminus in RNA molecules have been shown to have broad applications in studying RNA structures, mapping RNA-protein interactions, and in vitro selection of catalytic RNAs. Yet, current technology is limited in the ability for synthesizing modified RNA molecules especially relatively large RNA molecules having 50 or more nucleotide bases. Thus, there currently exists a need for producing modified RNA molecules, especially of relatively large size, whose 5′-terminus contains a thiol group.

### SUMMARY OF THE INVENTION

The present invention pertains to methods for forming an RNA molecule that contains a 5′-terminal thiol group. There are at least two general protocols disclosed in the instant invention that leads to the formation of a 5′-thiol-RNA molecule. One synthetic pathway leads to the formation of 5′-GSMP which is subsequently used as a substrate for an RNA polymerase forming a 5′-thiol-RNA molecule. The method requires an additional step of dephosphorylation of 5′-GSMP-RNA to produce 5′-HS-G-RNA. Another synthetic pathway leads to the formation of 5′-HS-PEG-GMP which in turn is also used as a substrate for an RNA polymerase. Generally, a nucleoside, such as guanosine, uridine, cytidine and adenosine can be used as the initial substrate in forming the modified RNA molecule. Preferably, guanosine is used as the initial substrate in forming the modified RNA molecule. The nucleoside is processed in such a manner as to render its 5′ terminus receptive for receiving a thiol group. The thiol group can then be added to the nucleoside creating a modified 5′-thiol-molecule. This nascent 5′-thiol molecule can then be subjected to transcription using an RNA polymerase, such as the T7 RNA polymerase, creating a 5′-thiol-RNA molecule. The presence of the polyethylene glycol (PEGₙ) linker may be important for bioconjugation of molecules. The number of PEGₙ polymer units can be from about 1 to about 20 PEG polymers, preferably from about 1 to about 10 PEG polymers, more preferably from about 1 to about 5 PEG polymers, and most preferably 2 and 4 PEG polymers. Other linker groups within the scope of the invention include, but are not limited to, alkyl groups, where R=hydrogen (H), HS—(CR₂)ₙ-GMP, and where “n” can be from about 1 to about 20, preferably, from about 1 to about 10, more preferably, from about 1 to about 5, and most preferably, 3; halogen substituted alkyl, where R=fluorine and where “n” can be from about 1 to about 20, preferably, from about 1 to about 10, more preferably, from about 1 to about 5, and most preferably, 3; Polyamine HS—[(CH₂)ₘNH]ₙ-GMP, where “m” can be from about 1 to about 10, preferably, from about 1 to about 5, and most preferably, from about 2 to about 4, and where “n” can be from about 1 to about 20, preferably, from about 1 to about 10, more preferably from about 1 to about 5, and most preferably, 2 and 4.

In particular, the invention pertains to 5′-modified guanosines that can be used as initiators for T7 RNA polymerase, to directly incorporate a free thiol to 5′-termini of RNA by in vitro transcription. In one embodiment, the initiator is O-[ω-sulfhydryl-bis(ethylene glycol)]-O-(5′-guanosine) monophosphate (5′-HS-PEG₂-GMP). In another embodiment, the initiator is O-[ω-sulfhydryl-tetra(ethylene glycol)]-O-(5′-guanosine) monophosphate (5′-HS-PEG₄-GMP). These initiators introduce a free thiol into 5′-end of RNA, and also provide a flexible PEG linker between HS group and RNA, which may be important for biocojugation of molecules. The detailed synthesis of 5′-deoxy-5′-thioguanosine-5′-monophophorothioate, O-[co-sulfhydryl-bis(ethylene glycol)]-O-(5′- guanosine) monophosphate, and O-[ω-sulfhydryl-tetra(ethylene glycol)]-O-(5′-guanosine) monophosphate is described herein. The 5′-Thiol labeled RNA molecules generated using the method of the invention were tested for their ability to conjugate with biological molecules. Three thiol-reactive biotin agents, biotin-PEG₃-iodoacetamide, biotin-HPDP, and biotin-PEG₃-Maleimide, were shown to couple with 5′-thiol of RNA molecules. The bioconjugation of maleimide-activated horseradish peroxidase with the 5′-sulfhydryl of RNA is also observed.

In one embodiment, 5′-deoxy-5′-thioguanosine-5′-mono-phophorothioate (GSMP) is synthesized starting from a guanosine molecule. Guanosine is treated with acetone and perchloric acid leading to the formation of 2′, 3′-isopropylideneguanosine. This 2′, 3′-isopropylideneguanosine is subsequently treated with methyltriphenoxyphosphonium iodide to give 2′, 3′-isopropylidene-5′-deoxy-5′-iodoguanosine. The 5′-iodo-guanosine derivative is deprotected using formic acid and subsequently treated with trisodium thiophosphate yielding the desired product, GSMP. GSMP can now be subjected to transcription using an RNA polymerase, for example T7 RNA polymerase, whose RNA product (5′-GSMP-RNA) is subsequently treated with alkaline phosphatase yielding a 5′-terminal thiol RNA molecule, 5′-HS-RNA.

In another embodiment, 5′-(polyethylene glycol)-5′-guanosine monophosphate (referred to as 5′-HS-PEG,-GMP, where n=1 to about 20, preferably, 1 to about 10, even more preferably, 1 to about 5, also referred to as O-[1-(12-mercapto-tetra(ethylene glycol)]-O-(5′-guanosine) monophosphate), is synthesized using phosphoramidite chemistry. In this embodiment, 2′, 3′-isopropylidene guanosine is treated with N,N-dimethylformamide dimethyl acetyl to form a protected guanosine. This protected guanosine is next reacted with (2-cyanoethyl-N,N-diisopropyl) chlorophosphoramidite to give a phosphoramidite that is subsequently coupled with tetra(ethylene glycol) monothioacetate, or other polyethylene gylcol monothioacetates, in the presence of 1H-tetrazole and is then deprotected to yield 5′-thiol-PEGₙ-GMP, where n=1 to about 20, preferably about 1 to about 10, more preferably, 1 to about 5. (“HS” herein will be interchangeably used with “thiol”). This 5′-thiol-PEG-GMP is then subjected to an RNA polymerase, such as the T7 RNA polymerase, yielding an RNA molecule which can then be treated with alkaline phosphatase giving 5′-HS-PEG-GMP-RNA, a 5′-terminal thiol RNA molecule.

In a preferred embodiment, 5′-deoxy-5′-thioguanosine-5′-monophophorothioate, O-[ω-sulfhydryl-bis(ethylene glycol)]-O-(5′-guanosine) monophosphate is synthesized using phosphoramidite chemistry. In yet another preferred embodiment, O-[ω-sulfhydryl-tetra(ethylene glycol)]-O-(5′-guanosine) monophosphate, is synthesized.

The present invention thus provides useful methods to efficiently modify the 5′-terminus of RNA. These methods have many potential applications for the analysis and detection of RNA, mapping RNA-protein interactions, in vitro selection of novel catalytic RNAs, and even gene array analysis. The methods of the invention can be used to thiol label RNA molecules that range in size from about 10 nucleotide bases to about 2000 nucleotide bases, preferably, from about 50 to about 1000 nucleotide bases, more preferably, from about 50 to about 600 nucleotide bases, and even more preferably from about 50 to about 300 nucleotide bases. The skilled artisan will appreciate that the methods of the invention can be used to produce RNA molecules where a nucleoside other than guanosine is used as a substrate using the appropriate RNA polymerase for each nucleoside. The double stranded DNA (dsDNA) can also be from about 10 to about 2000 base pairs, preferably, from about 50 to about 1000 base pairs, more preferably, from about 50 to about 600 base pairs, and even more preferably from about 50 to about 300 base pairs.

### BRIEF DESCRIPTION OF DRAWINGS

FIG. 1 illustrates the synthesis of 5′-deoxy-5′-thioguanosine-5′-mono-phosphorothioate;

FIG. 2 illustrates the synthesis of 5′-thiol-PEG-GMP, wherein (a) is Me₂NCH(OMe)₂, DMF, 50° C.; (b) is CIP (NPrⁱ₂)(OCH₂CH₂CN), NPrⁱ₂Et, CH₂Cl₂, 0° C.; (c) is H(OCH₂CH₂)₄SCOCH₃, 1H-tetrazole, MeCN, t-BuOOH; (d) is (1) 60% HCOOH/H₂O, (2) NH₃/MeOH, HS—CH₂CH₂OH;

FIG. 3 is a schematic diagram of the preparation of sulfhydryl incorporated RNA;

FIG. 4 is photograph of an autoradiogram of a streptavidin gel-shift analysis of transcription products following incubation with iodoacetyl-PEG-Biotin;

FIG. 5 illustrates the reactions of 5′-HS-RNA with thiol-reactive reagents;

FIG. 6 is a schematic diagram illustrating the synthesis of 5′-HS-PEGₙ-GMP 18a, 18b, and 18c^{a}

^{a}(a) acetone, 70% HClO₄; (b) Me₂NCH(OMe)₂, DMF, 55° C.; (c) ClP(NPrⁱ₂)(OCH₂CH₂CN), NPrⁱ₂Et, CH₂Cl₂, 0° C.; (d) (1) H(OCH₂CH₂)ₙSCOCH₃, 1H-tetrazole, MeCN, (2) t-BuOOH; (e) 60% HCOOH/H₂O; (f) NH₃/MeOH, HS—CH₂CH₂OH;

FIG. 7 is a schematic diagram illustrating an alternative synthetic route for 5′-HS-PEG₄-GMP 18C^{a}

^{a}(a) ClP(NPrⁱ₂)(OCH₂CH₂CN), NPrⁱ₂Et, CH₂Cl₂, 0° C.; (b) (1) 6, 1H-tetrazole, MeCN, (2) t-BuOOH; (c) 60% HCOOH/H₂O; (d) NH₃/MeOH, HS-CH₂CH₂OH;

FIG. 8 is a schematic diagram illustrating the synthesis of 5′-deoxy-5′-thioguanosine-5′-monophosphorothioate 22^{a}

^{a}(a) methyltriphenoxy-phosphonium iodide, THF; (b) 50% HCOOH, 3 days; (c) trisodium thiophosphate, water, 3 days;

FIG. 9 is a schematic diagram of enzymatic incorporation to yield 5′-sulfhydryl modified RNA and their subsequent detection by conjugation with thiol-reactive reagents;

FIG. 10 illustrates the chemical structures of thiol-reactive biotin agents;

FIG. 11 is a photogragh of an autoradiogram of RNAs transcribed in the presence and absence of 5′-HS-PEGₙ-GMP as initiator nucleotides, and incubated with maleimide-activated horseradish peroxidase prior to electrophoresis;

FIG. 12(A) is a photograph of an autoradiogram of RNAs transcribed using various ratios of GTP: 5′-HS-PEG₂-GMP and incubated with maleimide activated horseradish peroxidase prior to electrophoresis;

FIG. 12(B) is a bar chart showing the quantitative analysis of transcription yield and incorporation efficiency of 10 b using maleimide-activated horseradish peroxidase to detect 5′-HS-PEG₂-GMP-initiated RNA;

FIG. 13 is a photograph of an autoradiogram of the streptavidin gel-shift analysis of transcription products (5′-GTP-RNA and 5′-HS-PEG₂-RNA) following an incubation with 15, 16, or 17. Lane 1-3: 5′-GTP-RNA; lane 4-8: 5′-HS-PEG₂-RNA; and

FIG. 14. is a photograph of an autoradiogram of the streptavidin gel-shift analysis of transcription products (5′-GTP-RNA and 5′-GSMP-RNA) following an incubation with 15, 16, or 17. Lane 1-3: 5′-GTP-RNA; lane 4-10: 5′-HS-G-RNA.

### DETAILED DESCRIPTION OF THE INVENTION

The present invention pertains to methods for forming an RNA molecule that contains a 5′-terminal thiol group. There are at least two protocols disclosed in the instant invention that lead to the formation of a 5′-thiol-RNA molecule. One synthetic pathway leads to the formation of 5′-GSMP which is used as a substrate for an RNA polymerase to form a 5′-thiol-RNA molecule. A second synthetic pathway leads to the formation of 5′-HS-PEG-GMP which in turn is also used as a substrate for an RNA polymerase forming a modified RNA molecule.

Generally, a nucleoside, for example guanosine, is used as the initial substrate in forming the modified RNA molecule. The nucleoside is processed in such a manner as to render its 5′ terminus receptive for receiving a thiol group. The thiol group, complexed with a phosphate group, can then be added to the nucleoside creating a modified 5′-thiol-base molecule. This nascent 5′-thiol-base molecule can then be incorporated into a newly formed RNA molecule by transcription using an RNA polymerase, such as the T7 RNA polymerase, creating a 5′-thiol-RNA molecule. The thiol modification of the RNA molecule can facilitate, for example, the introduction of markers such as fluorophores onto individual RNA base residues as well as a residue in a fully, or partially, transcribed RNA molecule. The addition of markers to these molecules enhances the ability to analyze various physiological and pathophysiological processes occurring in a given cell system.

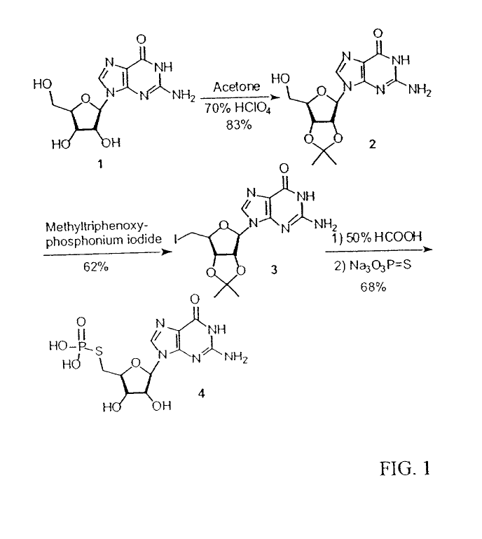

In one embodiment of the present invention, the synthesis of 5′-GSMP-RNA depends upon the formation of 5′-deoxy-5′-thioguanosine-5′-mono-phosphorothioate (GSMP) (4). The synthesis of (GSMP) (4) itself is depicted in FIG. 1 which illustrates the various reaction steps. Once GSMP (4) is formed, it is reacted with an RNA polymerase yielding a product that is subsequently treated with alkaline phosphatase to give 5′-HS-RNA, a 5′-terminal thiol RNA molecule. Representative reactions forming intermediates and their respective conditions for this embodiment are provided in greater detail immediately below.

In one embodiment, guanosine (1) is used as the starting nucleoside. Guanosine (1) is treated with acetone to form 2′, 3′-isopropylideneguanosine (2). This protected guanosine in the next reaction forms 2′, 3′-isopropylidene-5′-deoxy-5′-iodoguanosine (3). The 2′, 3′-isopropylidene-5′-deoxy-5′-iodoguanosine (3) is then deprotected yielding a crude product, namely GSMP (4).

An example of synthesizing the protected guanosine, 2′, 3′- isopropylidene-guanosine (2), from guanosine (1) involves the addition of approximately 70% perchloric acid (approximately 4.1 ml, 47.54 mmol) to a suspension of guanosine (approximately 10 g, 35.31 mmol) dissolved in 600 ml of acetone. After 70 minutes, concentrated ammonium hydroxide (approximately 6.7 ml, 49.79 mmol) is added to the reaction mixture and cooled down using an ice-water bath, or the like. The solid, 2′, 3′-isopropyl-ideneguanosine (2), is then filtered out and dried over a vacuum, yielding approximately 9.5 g (or 83.2%). ¹H-NMR (400 MHz, DMSO-d₆): δ10.67 (b, 1H, NH), 7.89 (s, 1H), 6.50 (b, 2H, NH2), 5.90 (d, J=2.7 Hz, 1H), 5.17 (dd, J=6.2 Hz), 5.03 (t, J=5.1 Hz, 1H, OH), 4.94 (dd, 11), 4.09 (ddd, 1H), 3.50 (m, 21), 1.49 (s, 3H, CH₃), 1.29(s, 3H, CH₃).

This 2′, 3 ′-hydroxl protected guanosine (2) is next reacted with methyltriphenoxy-phosphonium iodide to give 2′, 3′-isopropylidene-5′-deoxy-5′-iodoguanosine (3). In one embodiment, 2′, 3′-isopropylidene-5′-deoxy-5′-iodoguanosine molecule (3) is formed by adding methyltriphenoxyphosphonium iodide (approximately 0.86 g, 1.91 mmol) to a cooled (approximately −78° C.) suspension of 2′, 3′-O-isopropylideneguanosine (approximately 0.41 g, 1.27 mmol) in tetrahydrofuran (approximately 20 ml). The mixture is allowed to warm to room temperature after approximately 10 minutes. After about 4 hours the excess methyltriphenoxyphosphonium iodide is destroyed by addition of approximately 1 ml of methanol. The solvent is subsequently removed by reduced pressure. The residue is suspended in a mixture of ethyl ether and hexane (about 1:1) and is filtered and washed thoroughly by the mixture of ethyl ether and hexane. The crude product, 2′, 3′- isopropylidene-5′-deoxy-5′-iodoguanosine molecule (3), is purified by flash chromatography (gradient of methanol/chloroform), and approximately 0.34 g (61.8%) is obtained. R_{f}=0.53 (chloroform/methanol=4:1); ¹H-NMR (300 MHz, DMSO-d₆): δ7.88 (s, 1H), 6.55 (b, 2H, NH₂), 6.01(d, 1H), 5.30(dd, 1H), 5.04(dd, 1H), 4.25(ddd, 1H), 3.35(m, 2H), 1.50(s, 3H), 1.31 (s, 1H). This 5′-iodo-guanosine derivative (3) can also be purified by using a silica-gel column to give a pure product with approximately 62% yield.

Next, the 2′, 3′-isopropylidene-5′-deoxy-5′-iodoguanosine molecule (3) is deprotected. Deprotection of the 5′-iodo-guanosine derivative (3) is accomplished by using approximately 50% aqueous formic acid. Following deprotection, trisodium thiophosphate is added to the deprotected molecule which leads to the crude desired product, i.e., 5′-deoxy-5′-thioguanosine-5′-monophosphorothioate (GSMP) (4).

Alternatively, 5′-deoxy-5′-thioguanosine-5′-monophosphorothioate (GSMP) (4) can be synthesized by adding trisodium thiophosphate (approximately 4.8 g, 26 mmol) to a suspension of 5′-deoxy-5′-iodoguanosine (approximately 2.83 g, 7.2 mmol) contained in about 140 ml of water. The reaction mixture is stirred for about 3 days at room temperature under argon atmosphere. After filtration, to remove any precipitate, the filtrate is evaporated under reduced pressure. The residue is dissolved in about 100 ml of water and the trisodium thiophosphate is subsequently precipitated. After trisodium thiophosphate is precipitated, it is removed efficiently by adding methanol to the crude aqueous reaction mixture. Subsequent reverse phase chromatography and elution with water affords the pure desired product, GSMP (4). This GSMP (4) molecule can be characterized by proton and phosphorus NMR, MS spectroscopy, and tested as a substrate for in vitro transcription. After removing the precipitate by filtration, the filtrate is evaporated and dissolved in a small amount of water and subjected to reverse phase chromatography (C₁₈). GSMP (4) is collected and dried by a lyophilizer yielding approximately 1.9 g. R_{f}=0.36 (i-PrOH:NH₃:H₂O=6:3:1). ¹HNMR (400 MHz, DMSO-d6+D₂O): δ7.82 (s, 1H), 5.63 (d, J=5.9 Hz, 1H), 4.28 (dd, J=3.9 Hz 1H), 4.08 (ddd, 2H), 2.83 (m, 2H). ³¹P NMR (D₂O): δ16.4 ppm. MS (ESI) m/z found 378 [M−H⁺] (calculated C₁₀H₁₄N₅O₇PS, 379).

In still another alternative, synthesis of GSMP (4) can be accomplished in the following manner: A suspension of 5′-deoxy-5′-iodo-2′, 3′-isopropylidene guanosine (approximately 2.88 g, 6.65 mmol) in about 50% aqueous formic acid (approximately 100 ml) is stirred for about 2.5 days and then evaporated. The crude product, 5′-deoxy-5′-iodo-guanosine, (about 2.83 g) without further purification, is used in the next step of the reaction. R_{f}=0.78 (i-propyl alcohol:NH₃:H₂O=6:3:1). To a suspension of 5′-deoxy-5′-iodo-guanosine (about 2.83 g, 7.2 mmol) in approximately 140 ml of water is added trisodium thiophosphate (about 4.8 g, 26 mmol) followed by stirring for about 3 days at room temperature under argon atmosphere. After filtration to remove any precipitate, the filtrate is evaporated under reduced pressure. The residue is then dissolved in approximately 100 ml of water and precipitated by the addition of about 200 ml of methanol. After removing the precipitate by filtration, the filtrate is evaporated and dissolved in a small amount of water and subjected to reverse phase chromatography. GSMP (4) is collected and dried by lyophilization (about 1.9 g, 68% for two steps). R_{f}=0.36 (isopropylalcohol:NH₃:H₂O=6:3:1). ¹HNMR (400 MHz, DMSO-d₆+D₂O): δ7.82 (s, 1H), 5.63 (d, J=, 5.9 Hz, 1H), 4.28 (dd, J=3.9 Hz 1H), 4.08 (ddd, 2H) ³¹P NMR (D₂O): δ16.4. Mass spectrum ESI: calculated 379, found 378 (a negative ion).

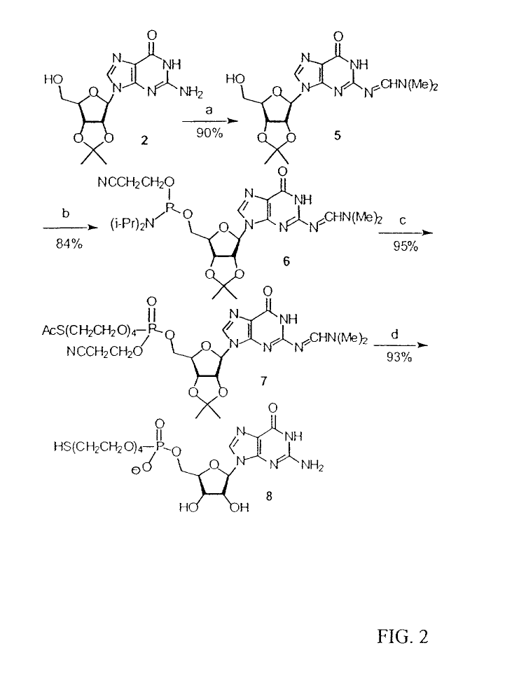

In another embodiment of the present invention, depicted in FIG. 2, a second pathway is employed to form a modified RNA molecule. This second pathway leads to the formation of 5′-HS-PEG-GMP-RNA which is synthesized using 5′-thiol-polyethylene gylcol-5′-guanosine monophosphate (8). The 5′-thiol-polyethylene gylcol-5′-guanosine monophosphate (8) itself is synthesized using phosphoramidite chemistry. The 2′, 3′-isopropylidene guanosine (2) molecule (mentioned previously in the synthesis of GSMP) is treated to form a protected guanosine, i.e., 2′, 3′-isopropylidene-2-dimethylform-amidine-guanosine (5). This protected guanosine (5) is next treated to form (2′, 3′-isopropylidene-2-N-dimethylformamidine guanosine) 2-cyanoethyl N,N-diisopropyl-amino phosphoramidite (6). This intermediate phosphoramidite (6) is then a reactant in a subsequent coupling reaction in order to yield the desired product 5′-thiol-PEG-GMP (8).

The synthesis of 2′, 3′-isopropylidene-2-dimethylformnamidine-guanosine (5) (an example of a protected guanosine) preferably involves the following steps: Obtain a solution of 2′, 3′-isopropylidene guanosine (2) (about 9.78 g, 30.2 mmol) and dimethylformamide dimethyl acetal (about 15 ml, 0.11 mol) in anhydrous DMF (about 100 ml) which is stirred for about 24 hours at about 55° C. under argon. The clear light-yellow solution that is produced is then subsequently evaporated under reduced pressure. The residue is stirred in approximately 40 ml of methanol leading to a white precipitate. Addition of ethyl acetate (about 100 ml) leads to more of a solid precipitate. The preparation is then cooled to approximately −20 ° C. and filtered. The solid is dried to give about 6.6 g of pure product. The resultant is then concentrated to about 20 ml. A white solid precipitate is filtered off, washed with ethyl acetate, and dried under vacuum to give about 2.2 g (total yield, around 90%) of 2′, 3′-isopropylidene-2-dimethylformamidine-guanosine (5). TLC (silica gel, AcOEt:MeOH=3:1), R_{f}=0.42. ¹HNMR data (300 MHz, DMSO-d₆): δ11.38 (s, 1H, 1-NH), 8.55 (s, 1H, CHNMe₂), 8.00 (s, 1H, 8-CH), 6.02 (d, J(H—H)=2.7 Hz, 1′-CH), 5.25 (dd, J(H—H)=2.7, 6.2 Hz, 1H, 2′-CH), 5.06 (t, J(H—H)=5.5 Hz, 1H, 5′-OH), 4.94 (dd, J(H—H)=2.7, 6.2 Hz, 1H, 3′-CH), 4.12 (dt, J(H—H)=2.7, 5.1 Hz, 1H, 4′-CH), 3.50 (m, 2H, 5′-CH₂), 3.14 (s, 3H, NCH₃), 3.02 (s, 3H, NCH₃), 1.52 (s, 3H, CH₃), 1.31 (s, 3H, CH₃).

The next step in the formation of 5′-HS-PEG-GMP involves reacting the protected guanosine (5), 2′, 3′-isopropylidene-2-dimethylformamidine-guanosine, with (2-cyano-ethyl-N,N-diisopropyl)-chlorophosphoramidite to form (2′, 3′-isopropylidene-2-N-di-methylformamidine guanosine) 2-cyanoethyl N,N-diisopropyl-amino phosphoramidite (6).

An example of the synthesis of this phosphoramidite (6), (2′, 3′-isopropylidene-2-N-dimethylformamidine guanosine) 2-cyanoethyl N,N-diisopropylamino phosphor-amidite, involves the addition of approximately 10 ml of N,N-diisopropylethyl amine, under argon atmosphere, to a suspension of the protected guanosine (5) (about 5.0 g, 13.2 mmol) in anhydrous dichloromethane (about 50 ml). The mixture is then cooled to 0° C. and 2-cyanoethyl diisopropylamino phosphorous chloride (about 5 ml, 21.4 mmol) is added dropwise affording an almost colorless solution. The reaction is completed after approximately 30 minutes. Approximately 500 ml of ethyl acetate is added to the reaction solution and cold water (about 20 ml) is carefully introduced. The aqueous layer is separated and the organic layer is washed with cold water (about 2×100 ml) and cold saturated NaCl solution (about 2×100 ml). The combined aqueous layers are extracted with ethyl acetate (about 2×150 ml). The combined organic layers are dried over anhydrous MgSO₄. A slight yellow oil is obtained after evaporation of the solvent. ¹H-NMR showed the existence of HP(O)(NPrⁱ₂)(OCH₂CH₂CN) that is formed by the hydrolysis of excess ClP(NPrⁱ₂)(OCH₂CH₂CN), which is separated by chromatography using a silica gel column and eluted with AcOEt:Et₃N (95:5) and AcOEt:MeOH:Et₃N (85:10:5). Upon evaporation under reduced pressure, a white foam solid, i.e., (2′, 3′-isopropylidene-2-N-dimethylformamidine guanosine) 2-cyanoethyl N,N-diisopropyl-amino phosphoramidite (6), is obtained in a yield of about 83.5% (about 6.37 g). TLC (silica gel): Rf=0.73 (AcOEt:MeOH:Et₃N=85:10:5); 0.64 (AcOEt:MeOH=4:1). ¹H NMR (300 MHz, CDCl₃): δ9.08 (br, 1H, 1-NH), 8.63 (s, 1H, CHNMe₂), 7.92, 7.88 (2s, 1H, 8-CH), 6.14 (dd, J(H—H)=2.7, 4.2 Hz, 1′-CH), 5.14 (m, 1H, 2′-CH), 4.99 (m, 1H, 3′-CH), 4.44 (m, 1H, 4′-CH), 3.80 (m, 4H, PN(CHMe₂)₂, OCH₂CH₂CN), 3.58 (m, 2H, 5′-CH₂), 3.20 (s, 3H, NCH₃), 3.12 (s, 3H, NCH₃), 2.68 (m, 2H, CH₂CN), 1.63 (s, 3H, CH₃), 1.40 (s, 3H, CH₃), 1.14 (m, 6H, NCHMe₂). ³¹P NMR (CDCl₃): δ124.5, 124.4.

The (2′, 3′-isopropylidene-2-N-dimethylformamidine guanosine) 2-cyanoethyl N,N-diisopropylamino phosphoramidite (6) that is formed is then coupled with tetra(ethylene glycol) monothioacetate in the presence of 1H-tetrazole and finally deprotected by using about 60% aqueous formic acid and ammonia-methanol solution, respectively, giving the desired product, namely 5′-thiol-PEG-GMP (8).

However, it should be noted that the reaction which converts phosphoramidite (6) to 5′-thiol-PEG-GMP (8) involves the formation of an intermediate compound, (2′, 3′-acetonide 2-N-dimethyl-formamidine guanosine) 2-cyanoethyl [12-thioacetyl-tetra(ethylene glycol)] phosphate (7).

The synthesis of this intermediate, (2′, 3′-acetonide 2-N-dimethylformamidine guanosine) 2-cyanoethyl [12-thioacetyl-tetra (ethylene glycol)] phosphate (7), involves taking a solution of tetra(ethylene glycol) monothioacetate (about 2.27 g, 9 mmol) in approximately 20 ml of anhydrous acetonitrile, and adding it to a solution of 1H-tetrazole (about 2.9 g, 41.4 mmol) which is contained in approximately 80 ml of anhydrous acetonitrile. (The synthesis of tetra (ethylene glycol) monothioacetate is described below.) A solution of (2′, 3′-isopropylidene-2-N-dimethylform-amidine guanosine) 2-cyanoethyl N,N-diisopropylamino phosphoramidite (6) (about 4.0 g, 7.0 mmol) in approximately 20 ml of acetonitrile is then added dropwise to the above formed solution. The reaction can be monitored by TLC (AcOEt:MeOH=4:1, R_{f}=0.44). More tetra (ethylene glycol) monothioacetate (about 0.76 g, 3 mmol) is added after about 0.5 hours to complete the reaction. Upon the disappearance of (2′, 3′-isopropylidene-2-N-dimethylformamidine guanosine) 2-cyanoethyl N,N-diisopropyl-amino phosphoramidite (6) in about 30 minutes, tert-butyl hydroperoxide (about 10 ml) is added. The mixture is stirred for about 30 minutes at approximately room temperature. After evaporation of the solvent under reduced pressure, the residue is dissolved in about 400 ml of ethyl acetate, washed with cold water (about 2×100 ml) and saturated NaCl (about 2×100 ml). The aqueous layer is extracted with ethyl acetate (about 2×100 ml) and the combined organic layer is then dried over anhydrous MgSO₄. After removal of the solvent, the residue is applied to a silica-gel flash column and eluted with AcOEt:MeOH (approximately 5-20%). Evaporation of the solvent gives the desired compound: (2′, 3′-acetonide 2-N-dimethylformamidine guanosine) 2-cyanoethyl [12-thioacetyl-tetra(ethylene glycol)] phosphate (7), as a white foam solid with approximately 95% yield (4.96 g). TLC (silica gel, AcOEt:MeOH=4:1): R_{f}=0.22. ¹H NMR (300 MHz, CDCl₃): δ9.57 (br, 1H 1-NH), 8.57 (s, 1H, CHNMe₂), 7.72, 7.71 (2s, 1H, 8-CH), 6.04 (t, J(H—H)=3.0 Hz, 1′-CH), 5.26 (m, 1H, 2′-CH), 5.03 (m, 1H, 3′-CH), 4.38 (m, 1H, 4′-CH), 4.30−4.10 (m, 6H, 3 CH₂O), 3.66−3.52 (m, 12H, 5 OCH₂, 5′-CH₂), 3.19 (s, 3H, NCH₃), 3.09 (s, 3H, NCH₃), 3.05 (m, 2H, CH₂S), 2.72 (m, 2H, CH₂CN), 230 (s, CH₃C(O)S), 1.59 (s, 3H, CH₃), 1.37 (s, 3H, CH₃). ³¹P NMR (CDCl₃): δ−1.65.

Tetra (ethylene glycol) monothioacetate, which is a reactant in the formation of (2′, 3′-acetonide 2-N-dimethylformamidine guanosine) 2-cyanoethyl [12-thioacetyl-tetra(ethylene glycol)] phosphate (7), can be formed by starting with a suspension of potassium thioacetate. This potassium thioacetate (about 17.2 g, 0.15 mol), contained in approximately 650 ml of acetone, is added to a solution of tetra (ethylene glycol) monotosylate (about 21.0 g, 60.3 mmol) which is in approximately 100 ml of acetone at about room temperature. (An example of synthesizing tetra (ethylene glycol) monotosylate is provided below.) The mixture is stirred for approximately 1 hour at about room temperature, and then refluxed for about 4 hours. A solid precipitate is produced and subsequently filtered off and the filtrate is then evaporated under reduced pressure. The residue is dissolved in ethyl acetate (about 150 ml) and washed with water (about 2×50 ml) and brine (about 2×50 ml). The aqueous solution was extracted with ethyl acetate (about 2×50 ml). The combined organic layers are dried over anhydrous MgSO₄ and evaporated under reduced pressure to give a slight yellow oil. The product, tetra (ethylene glycol) monothioacetate, is purified by using a silica-gel column eluting it with hexane:AcOEt (6:1) to give approximately 8.75 g of the desired product (about 96%). TLC (silica gel, hexane:AcOEt=1:4): R_{f}=0.24. ¹H NMR (CDCl₃): d3.73 (t, J(H—H)=4.6 Hz, 2H, CH₂OH), 3.64−3.56 (m, 4H, 2 CH₂O), 3.10 (t, J(H—H)=6.1 Hz, 2H, SCH₂), 2.35 (s, 3H, CH₃), 2.08 (s, 1H, OH).

Synthesis of tetra(ethylene glycol) monotosylate can be performed as follows: To a solution of tetra(ethylene glycol) (about 100 ml, 0.58 mol) and anhydrous pyridine (about 40 ml, 0.50 mol), both of which are in approximately 200 ml of anhydrous dichloro-methane,p-toluenesulfonyl chloride (about 19.1 g, 0.10 mol, which is in about 100 ml of dichloromethane), is added dropwise. The mixture is stirred at approximately room temperature for about 20 hours. The reaction mixture is washed with cold water (about 2×100 ml) and saturated NaCI (about 2×100 ml). The aqueous solution is extracted with dichloromethane (about 2×150 ml) and the combined organic layers are dried over anhydrous MgSO₄. Upon evaporation under reduced pressure, a slight yellow oil becomes apparent. The product, tetra(ethylene glycol) monotosylate, is purified by a silica gel column eluted using a gradient of CH₂Cl₂/MeOH (approximately 0-5%) to give a colorless oil of about 31.3 g (90%). Data for tetra(ethylene glycol) monothioacetate: yield=96%. R_{f}=0.24 (silica gel, hexane:ethyl acetate=1:4). ¹H NMR (300 MHz, CDCl₃): δ3.73 (t, J=4.6 Hz, 2H), 3.64−3.56 (m, 4H), 3.10 (t, J=6.1 Hz, 2H), 2.35 (s, 3H), 2.08 (s, 1H).

After forming the completely protected 2′, 3′-acetonide 2-N-dimethylformamidine guanosine) 2-cyanoethyl [12-thioacetyl-tetra(ethylene glycol)] phosphate (7), approximately 3.87 g (5.19 mmol) of this molecule (7) is dissolved in approximately 60% formic acid (about 100 ml), and the solution is stirred for about 3 days at approximately room temperature, leading to a completely deprotected 2′, 3′-isopropylidene and 2-N-dimethylformamidine groups forming 2-cyanoethyl 5′-guanosine [12-thioacetyl tetra(ethylene glycol)] phosphate. Removal of the solvent under reduced pressure and co-evaporation with methanol twice affords a product that is used for the next reaction in forming 5′-thiol-PEG-GMP (8) without the need for further purification. An analytical amount of 5′-thiol-PEG-GMP (8) is obtained by employing a silica-gel flash column and eluting with AcOEt:MeOH (3:1). ¹HNMR (300 MHz, DMSO-d₆): δ8.46 (s, 1H, NH), 7.84 (s, 1H, 8-CH), 6.75 (br, 2H, NH₂), 5.75 (d, J(H—H)=5.6 Hz, 1′-CH), 4.44 (m, 1H, 2′-CH), 4.20−3.40 (m, 20 H, 3′-CH, 4′-CH, 5′-CH₂, 8 CH₂O), 3.00 (m, 2H, CH₂S), 2.88 (m, 2H, CH₂CN), 2.34 (s, 3H, CH₃C(O)S). ³¹P NMR (DMSO-d₆): δ3.2.

An alternative synthesis of O-[1-(12-mercapto-tetra(ethylene glycol))]-O-(5′-guanosine) monophosphate (8) (also referred to as 5′-thiol-PEG-GMP) is disclosed. The tetra(ethylene glycol) monothioacetate (about 1.5 g, 2.3 mmol) is dissolved in methanol (about 40 ml) under argon atmosphere. An excess of mercaptoethanol (about 2 ml, 28.5 mmol) is added, followed by the addition of methanolic ammonia (about 7N solution, 20 ml). The mixture is stirred at approximately 55° C. for about 1 day. After removal of the solvent under reduced pressure and co-evaporated with methanol (about 3×25 ml), the residue is washed with ethyl acetate to remove mercaptoethanol. The solid is dried under vacuum and is then applied to a C₁₈-reverse phase HPLC column, eluting the column using water and water-methanol (approximately 10-50%). The collected solution is evaporated under reduced pressure to remove any organic solvent and the aqueous solution is lyophilized to give a solid of 5′-thiol-PEG-GMP (8), yielding about 1.22 g (93%). TLC (silica gel, ⁱPrOH:NH₃:H₂O=7:1:2): R_{f}=0.45. ¹H NMR (300 MHz, D₂O): δ8.11 (s, 1H, 8-CH), 5.75 (d, J(H—H)=5.5 Hz, 1H, 1′-CH), 4.58 (t, J(H—H)=5.1 Hz, 1H, 2′-CH), 4.30 (t, J(H—H)=4.5 Hz, 2H, 3′-CH), 4.14 (m, 1H, 4′-CH), 3.93 (m, 2H, 5′-CH₂), 3.74 (m, 2H, CH₂OP), 3.45−3.41 (m, 12H, 6 OCH₂), 2.52 (s, 1H, SH), 2.48 (t, J(H—H)=6.3 Hz, 2H, CH₂S). ³¹P NMR (D₂O): δ1.30. Mass spectrum (ESI) m/e, [M+H]⁺=556.1 (cal. 555).

By techniques described above, two 5′-terminal thiol molecules have been separately formed, namely, GSMP (4) and 5′-thiol-PEG-GMP (8). These two products can now separately be subjected to RNA polymerization (“transcription”) using an RNA polymerase, such as T7 RNA polymerase, in the presence of dsDNA, and alkaline phosphatase in order to yield a 5′-thiol-RNA molecule. In the presence of RNA polymerase, dsDNA, GTP, ATP, UTP, CTP and either GSMP (4) or 5′-thiol-PEG-GMP (8), under conditions suitable for transcription well known to those of ordinary skill in the art, an RNA molecule is synthesized incorporating either GSMP or 5′-thiol-PEG-GMP, depending upon which one is used as a substrate. The nascent RNA molecule is then subjected to alkaline phosphatase treatment which removes the terminal phosphate group leading to a 5′-HS-RNA molecule.

Transcription reactions are well known to those of ordinary skill in the art and are generally carried out using 20 U of RNA polymerase, such as T7 RNA polymerase, in the presence of 1 mM each GTP, ATP, CTP and UTP, 10 μg of a DNA template, 10 μCi α-³²P-ATP, 4 mM spermidine, 0.05% Triton X-100, 12 mM MgCl₂ and 40 mM Tris buffer (pH 7.5) at 37° C. in a total 200 μl solution.

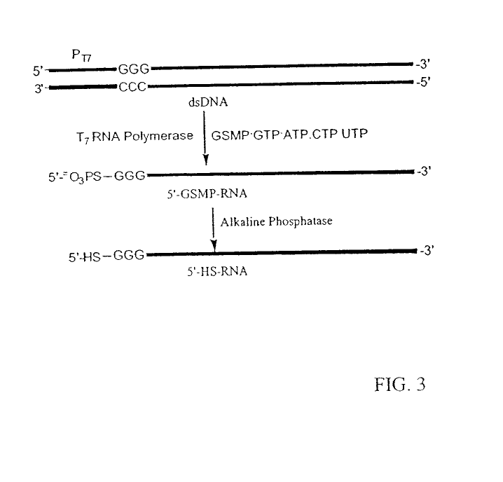

In one embodiment, a 222-mer double-stranded (ds) DNA containing a T7 promoter is used as the template for in vitro transcription (FIG. 3). Transcription reactions are performed using a T7 RNA polymerase in the presence of [γ-³²P]-ATP and 5′-deoxy-5′-thio-guanosine-5′-monophosphorothioate (GSMP) with a ratio of GSMP:GTP=8:1 or 4:1. The 5′-GSMP-RNA is purified by employing a denaturing polyacrylamide gel electrophoresis procedure. The gel purified 5′-GSMP-RNA is dephosphorylated by alkaline phosphatase to yield 5′-HS-RNA.

In another embodiment, a 196 nucleotide RNA is synthesized by runoff transcription in the presence of GMPS, GSMP (4) or 5′-HS-PEG-GMP (8) with a ratio of GMPS:GTP:ATP:CTP:UTP=8:1:1:1:1 mM. (GMPS is a molecule that is commercially available, for example through USB.) The newly formed modified RNA molecules, for example 5′-GSMP-RNA, can be incubated with 10 units of alkaline phosphatase in New England Biolab buffer “3” at 37° C. for 3 hours and stopped by the addition of 10 μl of 200 mM EGTA for 10 minutes at 65° C. The RNA can be then recovered by ethanol precipitation. To mark (or label) the RNA product, biotin can be used where the 5′-HS-RNA, 5′-GMPS-RNA or 5′-HS-PEG-RNA is reacted with PEG-iodoacetyl biotin (from Pierce) in 10 mM HEPES (pH 7.7) and 1 mM EDTA at room temperature for 2 hours. The 5′-Biotin-RNAs are resuspended in 20 μl of pure water and stored at −20° C. A 2 μl aliquot of 5′-Biotin-RNA is incubated with 10 μg of streptavidin in the binding buffer (20 mM HEPES, pH 7.4, 5.0 mM EDTA and 1.0 M NaCl) at room temperature for 20 minutes prior to mixing with 0.25 volumes of formamide loading buffer (90% formamide; 0.01% bromophenol blue and 0.025% xylene cyanol). The biotinylated RNA products are resolved by electrophoresis using 7.5 M urea/8% polyacrylamide gels. The fraction of product formation relative to total RNA at each lane can be quantitated with a Molecular Dynamics Phosphorlmager.

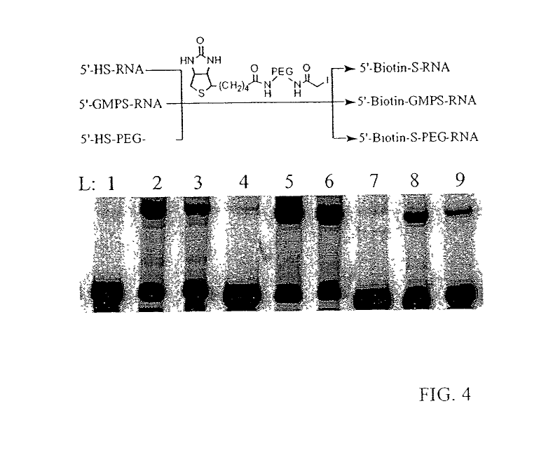

The efficiency of transcriptional incorporation of a marker with a modified RNA molecule, whose 5′-terminal possesses a thiol group, can be assessed using, for example, a gel-shift assay. To illustrate this point, 5′-thiol-RNA molecules were prepared by methods analogous to that described above using guanosine-5′-monophosphoro-thioate (GMPS) or 5′-HS-PEG-GMP (8) or GSMP (4). Using these substrates, 5′-GMPS-RNA, 5′-HS-PEG-GMP-RNA and 5′-HS-RNA were synthesized. These 5′-thiol-RNA molecules were then complexed with biotin, as described above. The 5′-GMPS-RNA, 5′-HS-PEG-GMP-RNA and 5′-HS-RNA, containing iodoacetyl-PEG-biotin, were analyzed using a streptavidin gel-shift assay as depicted in FIG. 4. The transcription reactions were performed using a 20 μg DNA template and 1.0 mM each NTP (ATP, CTP, GTP, UTP) under standard conditions. Alterations in the standard conditions were as follows: lane 1, 8 mM GSMP without streptavidin; lane 2, 8 mM GSMP with streptavidin; lane 3, 6 mM GSMP with streptavidin; lane 4, 8 mM GMPS without streptavidin; lane 5, 8 mM GMPS with streptavidin; lane 6, 6 mM GMPS with streptavidin; lane 7, 8 mM 5′-HS-PEG-GMP without streptavidin; lane 8, 8 mM 5′-HS-PEG-GMP with streptavidin; and lane 9, 4 mM 5′-HS-PEG-GMP with streptavidin. The biotinylated RNA can complex with streptavidin and the mobility of the 5′-biotin-RNA:streptavidin complex through the gel will be retarded relative to unbiotinylated RNA. In FIG. 4, GSMP (4) (lane 2 & 3) is demonstrated as being equally as good of an initiator for T7 RNA polymerase as is GMPS (lane 5 & 6). The total yield is 55% (three steps) for GSMP (4) (lane 2) and 57% (two steps) for GMPS (lane 5). However, 5′-HS-PEG-GMP (8) is not as good of an initiator when compared to GSMP (4) for T7 RNA polymerase (lanes 8). The average incorporation efficiency of GSMP is over 80% for each step with a GSMP:GTP ratio of 8:1 for transcription. If the ratio of GSMP:NTP is increased to 16:1, the incorporating yield will be significantly enhanced (data not shown). The 5′-GMPS-RNA can only react with haloacetamide (Br, I) and pyridyl disulfide agents, but the 5′-HS-RNA can react with any thiol-reactive agent.

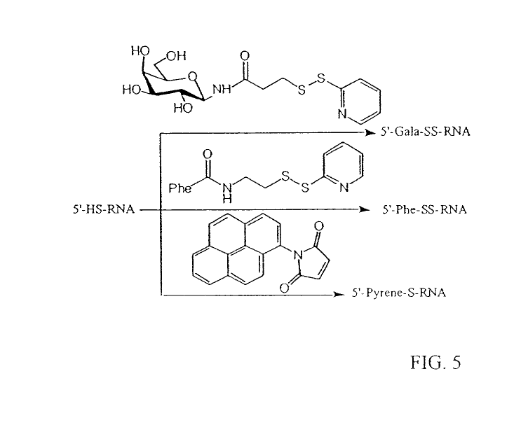

Furthermore, the derivatization of 5′-HS-RNA with three different thiol-reactive functional agents: iodo-acetamidyl-biotin, phenylanaline-pyridyl disulfides and β-galactose-pyridyl disulfides along with pyrene-maleimide was assessed (FIG. 5). The coupling of 5′-thiol-RNA with these reagents is essentially quantitative. The quantitative analysis for the coupling reactions of 5′-HS-RNA with pyridyl disulfide reagents was determined using the concentration of the pridine-2-thione released by measuring the absorbance at 343 nm, and quantitation for pyrene-maleimide was determined by the Molecular Dynamics Phosphorlmager.

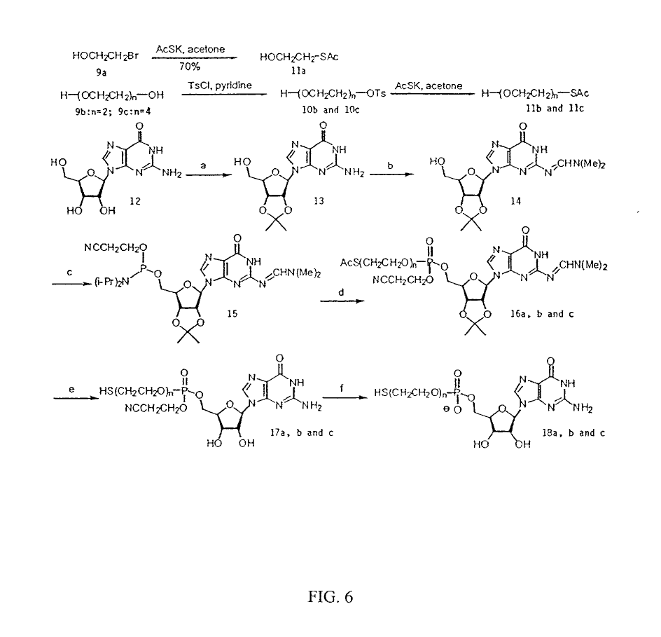

In a preferred embodiment, the synthesis and characterization of 5′-HS-PEG₂-GMP and 5′-HS-PEG₄-GMP are described, as shown in FIG. 6. The O-[ω-sulfhydryl-di(ethylene glycol)]-O-(5′-guanosine) monophosphate (18b) and O-[ω-sulfhydryl-tetra(ethylene glycol)]-O-(5′-guanosine) monophosphate (18c) were synthesized by phosphoramidite chemistry. The synthetic strategy was to initially synthesize 5′- phosphoramidite-2′, 3′-O,O-isopropylidene-2-N-(N′,N′-dimethylaminomethylene)-guanosine (15), following which, the free hydroxyl group of co-thioacetate-poly(ethylene glycol) compounds (11a, 11b and 11c) were coupled with 5′-phosphoramidite-2′, 3′-O,O-isopropylidene-2-N-(N,N′-dimethylaminomethylene)-guanosine (15) in the presence of 1H-tetrazole (FIG. 6). Different lengths of PEG linkers were incorporated at the 5′-phosphate of guanosine depending upon the specific version of ω-thioacetate-poly(ethylene glycol) compounds (11a-c) chosen. The polyethylene glycols (PEGs) were chosen as linkers because the flexibility that they provide, and because they reduce steric hindrance effects. PEG-containing GMP nucleotides are incorporated less efficiently as initiator nucleotides as the length of the PEG linker increases (Seelig et aL (1999) Bioconjugate Chem. 10, 371-378), therefore, the competing demands of linker flexibility with incorporation efficiency was balanced.

A large excess of ethylene glycol, or di-or tetra(ethylene glycol) was reacted with p-toluenesufonyl chloride in pyridine and then reacted with potassium thioacetate to afford ω-thioacetate-poly(ethylene glycol) compounds (11a, 11b, or 11c). Guanosine (12) was treated with acetone and 70% perchloric acid at room temperature to give 2′, 3′-O,O-isopropylideneguanosine (13) in 83% yield. The 2′, 3′-O,O-isopropylideneguanosine (13) was reacted with N, N-dimethylformamide dimethyl acetal in methanol to yield 2-N-(N′, N′-dimethylaminomethylene)-2′, 3′-O,O-isopropylidene guanosine (14) in 93% yield. The reaction of 2-N-(N′, N′-dimethylaminomethylene)-2′, 3′-O,O-isopropylidene guanosine (14) with (2-cyanoethyl-N, N-diisopropyl) chlorophosphoramidite yielded phosphoramidite (15) that was coupled subsequently with ω-thioacetate-poly(ethylene glycol) compounds (11a, 11b or 11c) in the presence of 1H-tetrazole to afford the fully protected compounds 2-cyanoethyl 5′-(2-N-dimethylformamidine-2′,3′-O,O-isopropylidene guanosine) (ω-thioacetylethyl) phosphate (16a) ,2-cyanoethyl 5′-(2-N-dimethylaminomethylene-2′-O,3′-O-isopropylidene- guanosine) [ω-thioacetyl di(ethylene glycol)] phosphate (16b), 2-cyanoethyl 5′-(2-N-dimethylaminomethylene-2′-O,3′-O-isopropylidene-guanosine) [ω-thioacetyl di(ethylene glycol)] phosphate (16c) (16a-c) in high yield: 74% for 2-cyanoethyl 5′-(2-N-dimethylformamidine-2′,3′-O,O-isopropylidene guanosine) (ω-thioacetyl ethyl) phosphate (16a), 81% for 2-cyanoethyl 5′-(2-N-dimethylaminomethylene-2′-0,3 ′-O-isopropylidene-guanosine) [ω-thioacetyl di(ethylene glycol)] phosphate (16b), and 95% for 2-cyanoethyl 5′-(2-N-dimethylaminomethylene-2′-O,3′-O-isopropylidene-guanosine) [ω-thioacetyl di(ethylene glycol)] phosphate (16c). The first deprotection of 2-cyanoethyl 5′-(2-N-dimethylformamidine-2′,3′-O,O-isopropylidene guanosine) (ω-thioacetylethyl) phosphate (16a), 2-cyanoethyl 5′-(2-N-dimethylaminomethylene-2′-O,3′-O-isopropylidene-guanosine) [ω-thioacetyl di(ethylene glycol)] phosphate (16b), 2-cyanoethyl 5′-(2-N-dimethylaminomethylene-2′-O,3′-O-isopropylidene-guanosine) [ω-thioacetyl di(ethylene glycol)] phosphate (16a-c) was effected by treatment with 60% aqueous formic acid at room temperature to yield 2-cyanoethyl 5′-guanosine (3-thioacetylethyl) phosphate (17a-c) in almost quantitative yield. Then crude products 2-cyanoethyl 5′-guanosine (3-thioacetylethyl) phosphate (17a), 2-cyanoethyl 5′-guanosine co-thioacetyl di(ethylene glycol)] phosphate (17b), 2-cyanoethyl 5′-guanosine [ω-thioacetyl tetra(ethylene glycol) phosphate (17c) were used for the next step of reaction without further purification. Finally, compounds 2-cyanoethyl 5′-guanosine (3-thioacetylethyl) phosphate (17a), 2-cyanoethyl 5′-guanosine [ω-thioacetyl di(ethylene glycol)] phosphate (17b), 2-cyanoethyl 5′-guanosine [ω-thioacetyl tetra(ethylene glycol)] phosphate (17c) were deprotected fully by treatment with ammonia-methanol solution in the presence of a large excess of 2-mecaptoethanol to afford O-[ω-mercapto-di(ethylene glycol)] O-(5′-guanosine) monophosphate (18b) and O-[ω-mercapto-tetra(ethylene glycol)] O-(5′-guanosine) monophosphate (18c) in their reduced forms. The free thiol group of each of the products reacted readily with the acrylonitrile side products that were formed from the deprotection of the cyanoethyl group to generate un-recoverable side-products via Michael addition (Kuijpers et al. (1993) Tetrahedron 49, 10931-10944). This problem was resolved by the addition of 2-mercaptoethanol in lieu of the dithiol, 1′-dipyridyl (DTDP) and dithiothreitol (DTT) alternatives. The acrylonitrile was captured by the sacrifice of the sulfhydryl of 2-mecaptoethanol. The final products (O-[ω-mercapto-di(ethylene glycol)] O-(5′-guanosine) monophosphate (18b) and O-[ω-mercapto-tetra(ethylene glycol)] O-(5′-guanosine) monophosphate (18c)) were purified by reverse-phase chromatography eluted with a gradient from water to 50% methanol in water. The identities of the 5′-sulfhydryl-modified guanosine monophosphates, O-[ω-mercapto-di(ethylene glycol)] O-(5′-guanosine) monophosphate (18b) and O-[ω-mercapto-tetra(ethylene glycol)] O-(5′-guanosine) monophosphate (18c), were confirmed by proton, carbon, and phosphorus NMR spectrometry and mass spectrometry.

A detailed synthesis protocol which shows various compounds and intermediates used in the synthesis of sulthydryl-modified guanosine monophosphates (18 a, b, c) is described as following passages and shown in FIG. 6.

The synthesis of di(ethylene glycol) monotosylate (10b), can be performed as follows: To a solution of di(ethylene glycol) (9b, 95 ml, 1.0 mol) and anhydrous pyridine (40.5 ml, 0.5 mol) in 250 ml of anhydrous dichloromethane was added dropwise a solution of p-toluenesulfonyl chloride (38.1 g, 0.2 mol) in 150 ml of dichloromethane. The mixture was stirred at room temperature overnight. The reaction solution was washed with cold water (2×100 ml) and brine (2×100 ml). The aqueous solution was extracted with dichloromethane (2×100 ml) and the combined organic layers were dried over magnesium sulfate. The solvent was evaporated under reduced pressure to give a slightly yellow oil. The crude product was purified by flash silica gel column chromatography using a gradient of dichloromethane/methanol (0-5%) to yield a colorless oil (42.3 g, yield=81.2%). TLC (silica gel, chloroform/methanol=95:5); R_{f}=0.42. ¹H NMR (CDCl₃): δ1.99 (s, 1H), 2.46 (s, 3H), 3.54 (t, J=4.5 Hz, 2H), 3.68 (m, 4H), 4.20 (t, J=4.6 Hz, 2H), 7.35 (d, J=8.4 Hz, 2H), 7.81 (d, J=8.4 Hz, 2H). ¹³C NMR (CDCl₃) 821.9, 61.8, 68.8, 69.5, 72.5, 128.2, 130.1, 133.1, 145.2. ESI Mass (m/z): calcd. for C₁₁H₁₆O₅S 260.1, found 283.3 [M+Na]⁺.

The synthesis of tetra(ethylene glycol) monotosylate (10c) can be performed as follows: To a solution of tetra(ethylene glycol) (9c, 100 ml, 0.58 mol) and anhydrous pyridine (40 ml, 0.50 mol) in 200 ml of anhydrous dichloromethane was added dropwise a solution ofp-toluenesulfonyl chloride (19.1 g, 0.10 mol) in 100 ml of dichloromethane. The mixture was stirred at room temperature for 20 hr. The reaction solution was washed with cold water (2×100 ml) and brine (2×100 ml). The aqueous solution was extracted with dichloromethane (2×100 ml) and the combined organic layers were dried over magnesium sulfate. Evaporation of solvent under reduced pressure gave a slightly yellow oil. The crude product was purified by flash silica gel column chromatography using a gradient of dichloromethane/methanol (0-5%) to give a colorless oil 31.3 g, yield=90%. TLC (silica gel, dichloromethane/methanol=95:5); R_{f}=0.43. ¹HNMR (CDCl₃) δ2.43 (s, 3H), 2.48 (s, 1H), 3.56-3.70 (m, 14H), 4.14 (t, J=4.8 Hz, 2H), 7.32 (d, J=8.4 Hz, 2H), 7.78 (d,J=8.4 Hz, 2H). ¹³CNMR(CDCl₃) δ21.5, 61.6, 68.6, 69.2, 70.6−70.2 (m), 72.3, 127.9, 129.7, 132.8, 144.7. ESI Mass (m/z): calcd. for C₁₅H₂₄O₇S 348.1, found 371.5 [M+Na]⁺.

The synthesis of 2-(thioacetyl)ethanol (11a) can be performed as follows: To a suspension of potassium thioacetate (11.4 g, 0.1 mol) in 500 ml of acetone was added dropwise 3.55 ml of bromoethanol (9a, 0.05 mol). The mixture was stirred at room temperature for 1 hr producing a white precipitate. The solid was filtered and the solvent was evaporated under reduced pressure. The residue was stirred in 100 ml of dichloromethane, and re-filtered and diluted to 500 ml with dichloromethane. The organic solution was washed with water (2×50 ml) and brine (2×50 ml). The aqueous wash solutions were re-extracted with dichloromethane (2×50 ml) and the combined organic layers were dried over magnesium sulfate and evaporated under reduced pressure to give an orange oil in almost quantitative yield and high purity. The crude product was purified through a silica gel column eluted with hexane/ethyl acetate (6:1) to afford 8.4 g of the desired product (70% yield). TLC (silica gel, hexane/ethyl acetate=1:2); R_{f}=0.56. ¹H NMR (CDCl₃) δ2.34 (s, 3H), 2.43 (br, 1H), 3.05 (t, J=6.1 Hz, 2H), 3.72 (t,J=6.1 Hz, 2H). ¹³C NMR (CDCl₃) 530.8, 32.2, 61.8, 196.7.

The synthesis of di(ethylene glycol) monothioacetate (11b) can be performed as follows: To a suspension of potassium thioacetate (17.2 g, 0.15 mol) in 650 ml of acetone was added a solution of di(ethylene glycol) monotosylate (10b) (15.6 g, 60.3 mmol) in 100 ml of acetone at room temperature. The mixture was stirred at room temperature for 1 hr and then refluxed for 4 hr. After cooling to room temperature, the solid was filtered off and the solution was evaporated under reduced pressure. The residue was dissolved in ethyl acetate (150 ml) and washed with water (2×40 ml) and brine (2×50 ml). The aqueous was solutions were re-extracted with ethyl acetate (2×50 ml) and the combined organic layers were dried over magnesium sulfate and evaporated under reduced pressure to give a yellow oil. The crude product was purified through a flash silica gel column eluted with hexane/ethyl acetate (6:1) to give 8.75 g of desired product, yield=88.8%. TLC (silica gel, hexane/ethyl acetate=1:2); R_{f}=0.38. ¹H NMR (CDCl₃, 400 MHz) δ2.30 (s, 3H), 2.51 (br, 1H), 3.06 (t, J=6.1 Hz, 2H), 3.59−3.52 (m, 4H), 3.68 (t,J=4.6 Hz, 2H). ¹³CNMR(CDCl₃, 400 MHz) δ29.0, 30.7, 61.8, 69.7, 72.2, 195.8. ESIMS calcd. for C₆H₁₂O₃S 164.1, found 187.0 M+Na]⁺.

The synthesis of tetra(ethylene glycol) monothioacetate (11c) can be performed as follows: To a suspension of potassium thioacetate (10.1 g, 88 mmol) in 650 ml of acetone was added a solution of tetra(ethylene glycol) monotosylate (10c) (15.4 g, 44 mmol) in 100 ml of acetone. The mixture was stirred at room temperature for 1 hr and then refluxed for 4 hr. After filtration, the solvent was evaporated under reduced pressure. The residue was dissolved in ethyl acetate (150 ml) and washed with water (2×50 ml) and brine (2×50 ml). The aqueous wash solutions were re-extracted with ethyl acetate (2×50 ml) and the combined organic layers were dried over magnesium sulfate and evaporated under reduced pressure to give a yellow oil. The crude product was purified through a flash silica gel column eluted with hexane/ethyl acetate (6:1) to afford 10.5 g of the desired product, yield=95%. TLC (silica gel, hexane/ethyl acetate=1:4); R_{f}=0.24. ¹HNMR (CDCl₃): δ2.26 (s, 3H), 2.95 (br, 1H), 3.01 (t, J=6.1 Hz, 2H), 3.56-3.64 (m, 14H). ¹³C NMR (CDCl₃) δ28.8, 30.6, 61.7, 69.8, 70.3, 70.4, 70.5, 70.7, 72.6, 195.6. ESI Mass (m/z) calcd. for C₁₀H₂₀O₅S 252.1, found 275.3 [M+Na]⁺.

The synthesis of 2′,3′-O,O-Isopropylidene guanosine (13) can be performed as follows: To a suspension of guanosine (8.7 g, 30.7 mmol) in 600 ml of acetone was added 3 ml of 70% perchloric acid. A clear colorless solution was formed after ca. 0.5 hr. The mixture was stirred at room temperature for 1 hr and 3 ml of concentrated NH₃.H₂O was added leading to a white precipitate. The solvent was evaporated under reduced pressure to afford a white solid that was stirred with 40 ml of H₂O for several hours, filtered, and washed with cold water. Drying over phosphorus pentoxide in vacuo gave a white solid (8.3 g, 83.6%). ¹HNMR (DMSO-d₆): δ1.30 (s, 3H), 1.50 (s, 3H), 3.52 (m, 2H), 4.10 (dt, J=3.0, 5.4 Hz, 1H), 4.95 (dd, J=3.0, 6.3 Hz, 1H), 5.05 (t, J=5.4 Hz, 1H), 5.17 (dd, J=2.7, 6.3 Hz, 1H), 5.90 (d, J=2.7 Hz, 1H), 6.50 (br, 2H), 7.90 (s, 1H), 10.66 (s, 1H).

The synthesis of 2-N,N-Dimethylaminomethylene-2′,3′-O,O-isopropylidene guanosine (14) can be performed as follows: A solution of 13 (9.78 g, 30.2 mmol) and dimethylformamide dimethyl acetal (15 ml, 0.11 mol) in anhydrous DMF (100 ml) was stirred under argon at 55° C. for one day. The clear light yellow solution was evaporated under reduced pressure. The residue was stirred in 40 ml of methanol leading to precipitation of a white solid. More product was precipitated by addition of ethyl acetate (100 ml). The mixture was cooled to −20° C. and filtered. The solid was dried over phosphorus pentoxide in vacuo to give 6.6 g of 2-N,N-Dimethylaminomethylene-2′,3′-O,O-isopropylidene guanosine (14). The mother liquor was concentrated to about 20 ml, white solid was formed again which was filtered and washed with ethyl acetate and dried over phosphorus pentoxide in vacuo to give 2.2 g more product (total 8.8 g, yield=90%). TLC (silica gel, ethyl acetate/methanol=3:1); R_{f}=0.42. ¹H NMR (DMSO-d₆): δ1.32 (s, 3H), 1.53 (s, 3H), 3.03 (s, 3H), 3.15 (s, 3H), 3.52 (m, 2H), 4.13 (m, 1H), 4.95 (dd,J=2.8, 6.4 Hz, 1H), 5.07 (t, J=5.4 Hz, 1H), 5.26 (dd, J=3.0, 5.8 Hz, 1H), 6.03 (d, J=2.8 Hz), 8.02 (s, 1H), 8.57 (s, 1H), 11.37 (s, 1H). ¹³C NMR (DMSO-d₆): δ25.2, 27.1, 34.6, 40.8, 61.4, 81.1, 83.5, 86.3, 88.5, 113.1, 119.8, 137.2, 149.5, 157.4, 157.6, 158.2. ESI Mass (m/z): calcd. for C₁₆H₂₂N₆O₅378.2, found 379.2 [M+H]⁺.

The synthesis of 2-cyanoethyl-N,N-diisopropylamino-5′-(2-N-dimethylaminomethylene-2′,3′-O,O-isopropylidene guanosine) phosphoramidite (15) can be performed as follows: To a suspension of 2-N,N-Dimethylaminomethylene-2′,3′-O,O-isopropylidene guanosine (14) (5.0 g, 13.2 mmol) in anhydrous dichloromethane (50 ml) was added 10 ml of NN-diisopropylethylamine in argon atmosphere. The mixture was cooled to 0° C. and 2-cyanoethyl N,N-diisopropylamino phosphorous chloride (5 ml, 21.4 mmol) was added dropwise. The reaction was completed after 30 min and diluted with ethyl acetate (500 ml). The solution was washed with cold water (2×100 ml) and brine (2×100 ml). The combined aqueous layers were re-extracted with ethyl acetate (2×150 ml) and the combined organic layers were dried over magnesium sulfate and filtered. Evaporation of solvent gave a slightly yellow residue that was applied to flash column chromatography (silica gel) and eluted with ethyl acetate/triethylamine (95:5) yield a white foam solid (6.37 g, 83.5% yield). TLC (silica gel, ethyl acetate/methanol/triethylamine=85:10:5); R_{f}=0.73. ¹HNMR (CDCl₃): a 1.14 (m, 12H), 1.38 (s, 3H), 1.61 (s, 3), 2.68 (m, 2H), 3.10 (s, 2H), 3.18 (s, 3H), 3.55 (m, 2H), 3.78 (m, 4H), 4.41 (m, 11), 4.97 (m, 1H), 4.97 (m, 1H), 5.12 (m, 1H), 6.11 (dd,J=2.7, 4.7 Hz, 1H), 7.85, 7.88 (2s, 1H), 8.61 (s, 11), 9.63 (br, 11). ¹³C NMR (CDCl₃) δ20.6 (dd, J=2.7, 7.3 Hz,), 24.8 (m), 27.5, 35.4, 41.7, 43.3 (dd, J=6.8, 12.3), 58.8 (dd, J=18.4, 22.6), 63.4 (dd, J=16.1, 21.4), 81.7 (d, J=2.3), 85.2 (d, J=7.7), 85.9 (t, J=9.2), 89.8 (d, J=8.4), 114.4 (d, J=1.5), 118.0 (d, J=12.3), 120.8, 136.8, 150.1 (d, J=1.5), 157.1, 158.3, 158.4. ³¹P NMR (CDCl₃): δ150.0, 151.1. ESI Mass (m/z): calcd. for C₂₅H₃₉N₈O₆P 578.3, found 579.1 [M+H]⁺.

The synthesis of 2-cyanoethyl 5′-(2-N-dimethylformamidine-2′,3′-O,O-isopropylidene guanosine) (ω-thioacetylethyl) phosphate (16a) can be performed as follows: To a solution of 1H-tetrazole (1.4 g, 20 mmol) in 40 ml of anhydrous acetonitrile was added a solution of 2-thioacetylethanol (11a) (0.6 g, 5 mmol) in 10 ml of anhydrous acetonitrile under argon atmosphere. A solution of 2-Cyanoethyl-N,N-diisopropylamino-5′-(2-N-dimethylaminomethylene-2′,3′-O,O-isopropylidene guanosine) phosphoramidite (15) (1.92 g, 3.32 mmol) in 7 ml of acetonitrile was then added dropwise and stirred at room temperature. More 2-thioacetylethanol (11a) (0.6 g, 5 mmol) was added after 0.5 hr leading to complete disappearance of 7 in ca. 0.5 hr. An 8.0 ml aliquot of ter/-butyl hydroperoxide was added and the mixture was stirred at room temperature for 0.5 hr. After evaporation of solvent under reduced pressure, the residue was dissolved in 250 ml of ethyl acetate, and washed with cold water (2×30 ml) and brine (2×50 ml). The combined aqueous layers were back-extracted with ethyl acetate (2×50 ml) and the combined organic layers were dried over magnesium sulfate. After removal of solvent under reduced pressure, the residue was applied to a silica gel flash column and eluted with ethyl acetate/methanol (0-20%). Evaporation of solvent gave the desired compounds as a white foam solid (1.51 g, 74.0%). TLC (silica gel, ethyl acetate/methanol=4:1); R_{f}=0.55. ¹H NMR (CDCl₃): δ1.40 (s, 3H), 1.63 (s, 3H), 2.32, 2.33 (2s, 3H), 2.76 (m, 2H), 3.05 (m, 2H), 3.10 (s, 3H), 3.22 (s, 3H), 4.05-4.35 (m, 6H), 4.42 (m, 1H), 5.05 (dd, J=3.6, 6.6 Hz, 1H), 5.29 (m, 1H), 6.10 (s, 1H), 7.82, 7.84 (2s, 1H), 8.60 (s, 1H), 9.05 (s, 1H). ³¹P NMR (CDCl₃): δ−2.3, −2.2. ESI Mass (m/z) calcd. for C₂₃H₃₂N₈O₉P 613.2, found 614.3 [M+H]⁺.

The synthesis of 2-cyanoethyl 5′-(2-N-dimethylaminomethylene-2′-O,3′-O-isopropylidene-guanosine) [ω-thioacetyl di(ethylene glycol)] phosphate (16b) can be performed as follows: To a solution of 1H-tetrazole (1.4 g, 20 mmol) in 40 ml of anhydrous acetonitrile was added a solution of di(ethylene glycol) monothioacetate (11b) (0.82 g, 5 mmol) in 10 ml of anhydrous acetonitrile under argon atmosphere. A solution of phosphoramidite (15) (2.0 g, 3.46 mmol) in 20 ml of acetonitrile was then added dropwise. After the mixture was stirred for 0.5 hr at room temperature, more di(ethylene glycol) monothioacetate (11b) (0.5 g, 3 mmol) was added and the mixture was stirred for an additional 0.5 hr. An 8.0 ml aliquot of tert-butyl hydroperoxide was added and the mixture was stirred at room temperature for 0.5 hr. After evaporation of solvent under reduced pressure, the residue was dissolved in 250 ml of ethyl acetate, and washed with cold water (2×30 ml) and brine (2×50 ml). The combined aqueous layers were back-extracted with ethyl acetate (2×50 ml) and the combined organic layers were dried over magnesium sulfate. After removal of solvent, the residue was applied to a silica gel flash column and eluted with ethyl acetate/methanol (5-15%). The pure product was obtained as a white foam solid (1.86 g, 81.9%). TLC (silica gel, ethyl acetate/methanol=4:1); R_{f}=0.25. ¹H NMR (CDCl₃): δ1.38 (s, 3H), 1.60 (s, 3H), 2.30, 2.31 (2s, 3H), 2.74 (m, 2H), 3.02 (m, 2H), 3.10 (s, 3H), 3.20 (s, 3H), 3.52-3.64 (m, 4H), 4.11-4.33 (m, 6H), 4.40 (m, 1H), 5.04 (dd, J=3.5, 6.4 Hz, 1H), 5.27 (dt, J=2.6, 6.6 Hz, 1H), 6.06 (dd, J=2.6, 4.0 Hz, 1H), 7.71, 7.72 (2s, 1H), 8.58 (s, 1H), 9.71 (br, 1H). ¹³C NMR (CDCl₃): δ19.8, 25.6, 27.4, 28.8, 30.8, 35.5, 41.8, 62.4, 67.1, 67.6, 69.6, 69.9, 81.0, 84.5, 89.8, 114.9, 116.8, 121.0, 136.9, 150.0, 157.2, 158.1, 158.4, 195.6. ³P NMR(CDCl₃): δ−0.7, −0.6. ESI Mass (m/z) calcd. for C₂₅H₃₆N₇O₁₀PS 657.2, found 658.1 [M+H]⁺.

The synthesis of 2-cyanoethyl 5′-(2-N-dimethylaminomethylene-2′-O,3′-O-isopropylidene-guanosine) [ω-thioacetyl di(ethylene glycol)] phosphate (16c) can be performed as follows: To a solution of 1H-tetrazole (2.9 g, 41.4 mmol) in 80 ml of anhydrous acetonitrile was added a solution of tetra(ethylene glycol) monothioacetate (11c) (2.27 g, 9 mmol) in 20 ml of anhydrous acetonitrile under argon atmosphere. A solution of 15 (4.0 g, 7.0 mmol) in 20 ml of acetonitrile was then added dropwise and the mixture was stirred at room temperature. More tetra(ethylene glycol) monothioacetate (11c) (0.76 g, 3 mmol) was added after 0.5 hr and the reaction was stirred for an additional 0.5 hr. A 10 ml aliquot of tert-butyl hydroperoxide was added and the mixture was stirred at room temperature for 0.5 hr. After removing solvent, the residue was dissolved in 400 ml of ethyl acetate and washed with cold water (2×50 ml) and brine (2×50 ml). The combined aqueous layers were re-extracted with ethyl acetate (2×50 ml) and the combined organic layers were dried over magnesium sulfate. After evaporation of solvent under reduced pressure, the residue was applied to a silica gel flash column and eluted with ethyl acetate/methanol (5-20%) to yield the desired product as a white foam solid (4.96 g, 95.0%). TLC (silica gel, ethyl acetate/methanol=4:1); R_{f}=0.22. ¹H NMR (CDCl₃): δ1.37 (s, 3H), 1.59 (s, 3H), 2.30 (s, 3H), 2.72 (m, 2H), 3.05 (m, 2H), 3.09 (s, 3H), 3.19 (s, 3H), 3.52-3.66 (m, 12H), 4.10-4.30 (m, 6H), 4.38 (m, 1H), 5.03 (m, 1H), 5.26 (m, 1H), 6.04 (t, J=3.0 Hz, 1H), 7.71, 7.72 (2s, 1H), 8.57 (s, 1H), 9.57 (br, 1H). ³¹P NMR (CDCl₃): δ−1.7. ESI Mass (m/z) calcd. for C₂₉H₄₄N₇O₁₂PS 745.3, found 746.1 [M+H]⁺.

The 2-cyanoethyl 5′-(2-N-dimethylaminomethylene-2′-O,3′-O-isopropylidene-guanosine) [ω-thioacetyl di(ethylene glycol)] phosphate (16c) was also prepared from the reaction of 2-N,N-dimethylaminomethylene-2′,3′-O,O-isopropylidene guanosine (14) and 2-Cyanoethyl N,N-diisopropylamino [ω-thioacetyl tetra(ethylene glycol)] phosphoramidite (19). To a suspension of 2-N,N-dimethylaminomethylene-2′,3′-O,O-isopropylidene guanosine (14) (189.2 mg, 0.5 mmol) in anhydrous dichloromethane (15 ml) was added a solution of 1H-tetrazole (210.1 mg, 3.0 mmol) in anhydrous acetonitrile (6 ml) and a solution of phosphoramidite (19) (226 mg, 0.5 mmol) in anhydrous acetonitrile (5 ml) under argon atmosphere. After stirring the mixture at room temperature for 0.5 hr, more 1H-tetrazole (210 mg, 3.0 mmol) and phosphoramidite (19) (290 mg, 0.64 mmol) were added. After the disappearance of compound guanosine (4), tert-butyl hydroperoxide (2 ml) was added and the mixture was stirred at room temperature for an additional 0.5 hr. After evaporation of solvents under reduced pressure, the oil residue was dissolved in dichloromethane (150 ml) and washed with water (2×20 ml) and brine (2×20 ml). The combined aqueous solutions were back-extracted with dichloromethane (2×20 ml) and the combined organic layers were dried over magnesium sulfate. After removal of solvent, the residue was applied to a flash column (silica gel) eluted with ethyl acetate/methanol (0-20%) to give the desired product as a white foam solid (344 mg, 92.8%).

The synthesis of 2-cyanoethyl 5′-guanosine (3-thioacetylethyl) phosphate (17a) can be performed as follows: The fully protected compound 2-cyanoethyl 5′-(2-N-dimethylformamidine-2′,3′-O,O-isopropylidene guanosine) (ω-thioacetylethyl) phosphate (16a) (1.53 g, 2.5 mmol) was dissolved in 40% formic acid (50 ml) and the solution was stirred at room temperature for 3 days to affect a complete deprotection of the 2′,3′-acetonide group. After removal of solvent under reduced pressure, the residue was co-evaporated with methanol twice to afford a crude product that was used for the next step of the reaction without further purification. An analytical amount of product was obtained by silica gel flash column eluted with ethyl acetate/methanol (3:1). ¹H NMR (D₂O): δ2.07, 2.09 (2s, 3H), 2.74 (m, 2H), 2.88 (m, 2H), 3.85-4.45 (m, 8H), 4.70 (m, 1H, partially overlapped by H₂O signal), 5.74 (d, J=5.4 Hz), 7.75 (s, 1H). ³¹P NMR (D₂O): δ−2.2, −2.1. ESI Mass (m/z) calcd. for C₁₇H₂₃N₆O₉PS 518.1, found 519.5 [M+H]⁺.

The synthesis of 2-cyanoethyl 5′-guanosine [ω-thioacetyl di(ethylene glycol)] phosphate (17b) can be performed as follows: The fully protected compound 2-cyanoethyl 5′-(2-N-dimethylaminomethylene-2′-O,3′-O-isopropylidene-guanosine) [ω-thioacetyl di(ethylene glycol)] phosphate (16b) (1.53 g, 2.5 mmol) was dissolved in 60% formic acid (50 ml) and the solution was stirred at room temperature for 3 days to deprotect the 2′, 3′-acetonide group. After evaporation of solvent under reduced pressure, the residue was co-evaporated with methanol twice to afford a crude product that was used for the next step of the reaction without further purification. ¹H NMR (D₂O): δ2.12, 2.14 (2s, 3H), 2.72 (m, 2H), 2.80 (dd, J=6.4 Hz, 12 Hz, 2H), 3.40 (dd, J=5.6, 12 Hz, 2H), 3.46 (m, 2H), 3.85-4.40 (m, 8H), 4.7 (m, 1H), 5.74 (d, J=5.4 Hz, 1H), 7.78 (s, 1H). ³¹P NMR(D₂O): δ−1.9, −1.8. ESI Mass (m/z)calcd. for C₁₉H₂₇N₆O₁₀PS 562.1, found 563.2 [M+H]⁺.

The synthesis of 2-cyanoethyl 5′-guanosine [ω-thioacetyl tetra(ethylene glycol)] phosphate (17c) can be performed as follows: The fully protected compound 2-cyanoethyl 5′-(2-N-dimethylaminomethylene-2′-O,3′-O-isopropylidene-guanosine) [ω-thioacetyl di(ethylene glycol)] phosphate (16c) (1.64 g, 2.5 mmol) was dissolved in 60% formic acid (50 ml) and the solution was stirred at room temperature for 3 days to affect a complete deprotection of the 2′, 3′-acetonide group. After evaporation of solvent under reduced pressure, the residue was co-evaporated with methanol twice to yield a crude product that was used for the next step of the reaction without further purification. An analytical amount of product was obtained by silica gel flash column eluted with ethyl acetate/methanol (3:1). ¹H-NMR (DMSO-d6): δ2.30 (s, 3H), 2.90 (m, 2H), 3.02 (m, 4H), 3.40-4.20 (m, 18 H. overlapped with water signal), 4.44 (m, 1H), 5.72 (d, J=6.4 Hz, 1H), 6.75 (br, 2H), 7.84 (s, 1H), 8.46 (s, 1H). ³¹P NMR (D₂O): J-1.6. ESI Mass (m/z) calcd. for C₂₃H₃₅N₆O₁₂PS 650.2, found 651.1 [M+H]⁺.

The synthesis of O-[ω-Mercapto-di(ethylene glycol)]-O-(5′-guanosine) monophosphate (18b) can be perfomed as follows: The crude product 2-Cyanoethyl 5′-guanosine [ω-thioacetyl di(ethylene glycol)] phosphate (17b) (1.4 g, 2.5 mmol) was dissolved in methanol (40 ml) under argon atmosphere and an excess of 2-mercaptoethanol (2 ml, 28.5 mmol) was added. To the above solution was added ammonia in methanol (7.0 N solution, 20 ml). The mixture was stirred at 55° C. for 1 day. After removal of solvent, the solid residue was washed with ethyl acetate to remove excess mercaptoethanol. The crude product was applied to a reverse phase column and eluted with water and water/methanol (10-50%). The collected fractions were evaporated under reduced pressure and the aqueous solution was lyophilized to yield a pure product (1.06 g, 88%). TLC (silica gel, isopropanol/ammonia/water=7:1:2); R_{f}=0.45. ¹H NMR (D₂O): δ2.40 (t, J=6.3 Hz, 2H), 2.51 (s, 1H), 3.33 (t, J=6.3 Hz, 2H), 3.37 (m, 2H), 3.69 (m, 2H), 3.90 (m, 2H), 4.12 (m, 1H), 4.30 (t, J=4.7 Hz, 2H), 4.59 (t, J=5.3 Hz, 1H), 5.71 (d, J=5.5 Hz, 1H), 7.95 (s, 1H). ³¹P NMR (D₂O): δ1.3. HRMS calcd. for C₁₄H₂₃O₉N₅PS 468.0954, found 468.0927.

The synthesis of O-[ω-Mercapto-tetra(ethylene glycol)]-O-(5′-guanosine) monophosphate (18c) can be performed as follows: The compound 2-cyanoethyl 5′-guanosine [ω-thioacetyl tetra(ethylene glycol)] phosphate (17c) (1.5 g, 2.3 mmol) was dissolved in methanol (40 ml) in argon atmosphere and an excess of 2-mercaptoethanol (2 ml, 28.5 mmol) was added. To the above solution was added ammonia in methanol (7.0 N solution, 20 ml). The mixture was stirred at 55° C. for 1 day. After evaporation of solvent under reduced pressure, the residue was co-evaporated with methanol (3×25 ml), and washed with ethyl acetate to remove excess mercaptoethanol. The solid product was dried in vacuo and then was applied to a reverse phase column and eluted with water and water/methanol (10-50%). The desired fractions were collected and evaporated under reduced pressure to remove organic solvent and the aqueous solution was dried by lyophilization to yield a cotton-like solid (1.22 g, 93%). TLC (silica gel, isopropanol/ammonia/water=7:1:2); R_{f}=0.45. ¹H NMR (D₂O): δ2.48 (t, J=6.3 Hz, 2H), 2.52 (s, 1H), 3.41-3.45 (m, 12H), 3.74 (m, 2H), 3.93 (m, 2H), 4.14 (m, 1H), 4.30 (t, J=4.5 Hz, 2H), 4.58 (t, J=5.1 Hz, 1H), 5.75 (d, J=5.5 Hz, 1H), 8.11 (s, 1H). ³¹P NMR (D₂O): δ1.3. HRMS calcd. for C₁₈H₃₁N₅O₁₁PS 556.1478, found 556.1479.

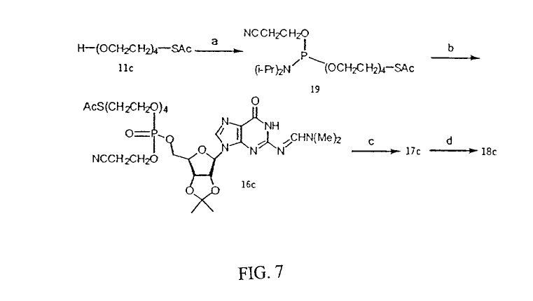

Alternatively, compound 2-cyanoethyl 5′-(2-N-dimethylaminomethylene-2′-O,3′-O-isopropylidene-guanosine) [ω-thioacetyl di(ethylene glycol)] phosphate (8c) also has been prepared from the reaction of protected guanosine (14) and 2-Cyanoethyl N,N-diisopropylamino [ω-thioacetyl tetra(ethylene glycol)] phosphoramidite (19) in a similar yield (FIG. 7).

The synthesis of 2-cyanoethyl N,N-diisopropylamino [ω-thioacetyl tetra(ethylene glycol)] phosphoramidite (19) can be performed as follows: To a solution of a ω-thioacetate-poly(ethylene glycol) compound (11c) (1.0 g, 3.96 mmol) in anhydrous dichloromethane (7 ml) was added N,N-diisopropylethylamine (2.0 ml, 11.5 mmol). The solution was cooled to 0° C. and then 2-cyanoethyl diisopropylamino phosphorous chloride was added dropwise. After stirring at 0° C. for 0.5 hr, ethyl acetate (100 ml) was added and the solution was washed with water (2×20 ml) and brine (2×20 ml). The combined aqueous layers were back-extracted with ethyl acetate (2×20 ml) and the combined organic layers were dried over magnesium sulfate. After removal of solvent, the residue was applied to a flash silica gel column eluted with heptane/ethyl acetate/triethylamine (80:15:5) to give a colorless oil (1.2 g, 67.0%). TLC (silica gel, ethyl acetate/heptane/triethylamine=10:9:1); R_{f}=0.57. ¹HNMR (CDCl₃): δ1.20 (d, J=6.6 Hz, 6H), 1.21 (d, J=6.6 Hz, 6H), 2.36 (s, 3H), 2.68 (t, J=6.5 Hz, 2H), 3.12 (t, J=6.5 Hz, 2H), 3.60-3.95 (m, 18H). ESI Mass (m/z) calcd. for C₁₉H₃₇N₂O₆PS 452.2, found 475.3 [M+Na]⁺.

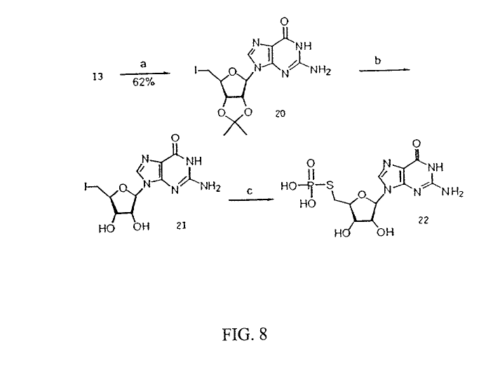

The synthesis of 5′-deoxy-5′-iodo-2′,3′-isopropylideneguanosine (20) is shown in FIG. 8, and can be performed as follows: Methyltriphenoxyphosphonium iodide (0.86 g, 1.91 mmol) was added to a cooled (−78° C.) suspension of 2′, 3′-O-isopropylidene guanosine (0.41 g, 1.27 mmol) in tetrahydrofuran (20 ml). The mixture was allowed to warm to room temperature after 10 minutes. After 4 hr the excess methyltriphenoxyphosphonium iodide was destroyed by addition of 1 ml of methanol and the solvent was removed under reduced pressure. The residue was suspended in a mixture of ethyl ether and hexane (1:1) and the solid was filtered and washed thoroughly by the addition of ethyl ether and hexane. The crude product was purified by flash chromatography (gradient of methanol/chloroformn) (0.34 g, 61.8%). R_{f}=0.53 (chloroform/methanol=4:1); ¹H-NMR (DMSO-d6): δ1.31 (s, 3H), 1.50 (s, 3H), 3.35 (m, 2H), 4.25 (m, 1H), 5.04 (dd, J=4.0 Hz, 8.4 Hz, 1H), 5.30 (dd, J=2.8 Hz, 1H), 6.01 (d, J=2.8 Hz, 1H), 6.55 (b, 2H), δ7.88 (s, 1H).

The synthesis of 5′-deoxy-5′-thioguanosine-5′-monophosphorothioate (GSMP) (22) can be performed using the protocol depicted in FIG. 8. Guanosine (12) was treated with acetone and 70% perchloric acid at room temperature for 70 minutes to give 2′, 3′-isopropylideneguanosine (5) with 83% yield and reacted with methyltriphenoxyphosphonium iodide (Dimitrijevich etal. (1979) J. Org. Chem. 44, 400-406) in THF to yield 2′, 3′-isopropylidene-5′-deoxy-5′-iodoguanosine (20) with 62% yield. 5′-Iodo-5′-deoxy-adenosine was synthesized by a similar procedure for 5′-iodo-5′-deoxyinosine synethsis (Hampton et al. (1969) Biochemistry 8, 2303-2311.

A suspension of 5′-deoxy-5′-iodo-2′,3′-isopropylidene guanosine (20) (2.88 g, 6.65 mmol) in 50% aqueous formic acid (100 ml) was stirred for 2.5 days and then the solvent was removed by evaporation. The crude deprotected product, 5′-deoxy-5′-iodoguanosine (21) (2.83 g) was used without further purification in the next reaction. R_{f}=0.78 (i-propyl alcohol/NH₃/H₂O=6:3:1). To a suspension of 5′-deoxy-5′- iodoguanosine (21) (2.83 g, 7.2 mmol) in 140 ml of water was added trisodium thiophosphate (4.8 g, 26 mmol). The reaction mixture was stirred for 3 days at room temperature under argon atmosphere. After filtration to remove any precipitate, the solvent was evaporated under reduced pressure. The residue was dissolved in 100 ml of water and precipitated by the addition of 200 ml of methanol. After removing the precipitate by filtration, the solvent was evaporated and the residue was dissolved in a small amount of water and subjected to reverse phase chromatography and eluted with water. The desired product was collected and dried by lyophilization (1.9 g, 68% for two steps). R_{f}=0.36 (isopropylalcohol/NH₃/H₂O=6:3:1). ¹H NMR (DMSO-d6+D₂O): δ2.83 (m, 2H), 4.08 (m, 2H), 4.28 (dd, J=3.9 Hz, 5.3 Hz, 1H), 5.63 (d, J=5.9 Hz, 1H), 7.82 (s, 1H); ³¹P NMR (DMSO-d6+D₂O): δ16.4. HRMS C₁₀H₁₅N₅O₇PS calcd. 380.0430, found 380.0482.

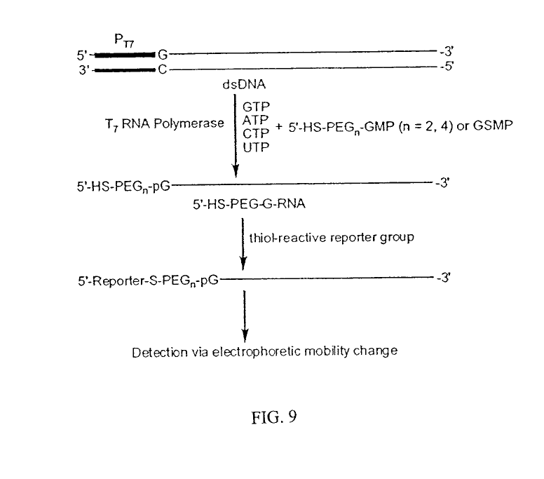

The 5′ terminal thiol molecules were used in RNA polymerase reactions to produce 5′-thiol modified RNA. The inventions pertains to the preparation of 5′-HS-PEG₂-GMP-RNA, 5′-HS-PEG₄-GMP-RNA, and 5′-HS-G-RNA, as shown in FIG. 9. The 5′-GTP-RNA, 5′-GSMP-RNA, and 5′-HS-PEG-GMP-RNA were prepared by run-off transcription in the presence of the four ribonucleotides or the four ribonucleotides supplemented with GSMP or 5′-HS-PEGₙ-GMP (18b or 18c) (FIG. 9). In general, the 222-base pair DNA template for in vitro transcription was generated by PCR from pC25 plasmid DNA (Zhang et al. (1997) Nature 390, 96-100). Transcription reactions were carried out with 4 μl of T7 RNA polymerase in the presence of 2 mM each NTP, 7.2 μg of DNA template, 10 μCi α-³²P-ATP, 4 mM spermidine, 0.05% Triton X-100, 12 mM MgCl₂, 20 mM DTT, and 40 mM Tris buffer (pH 7.5) in a total 200 μl reaction at 37° C. for 3 hours. A 4 μl aliquot of 0.5 M EDTA (pH 7.4) was added to dissolve the white Mg²⁺-pyrophosphate precipitate and 80 μl of formamide dye was added, and then loaded on an 8% polyacrylamide gel. RNA was purified through an 8% polyacrylamide [29:1 acrylamide:bis(acrylamide)]/8 M urea gel. RNA was visualized by UV shadowing and excised from the gel. The gel slice was crushed and soaked overnight in TE buffer (10 mM Tris-HCl, pH 7.5, 1 mM EDTA, and 250 mM NaCl) at 4° C. to elute the RNA. After filtering the soaking solution, RNA was recovered by ethanol precipitation and the pellet was dissolved in 10-50 μl of ddH₂O. The sequence of the full length RNA is as follows: 5′-GGG AGA GAC CUG CCA UUC ACG CUG GAU AAA ACU UCA CAG CCA UAC GUW GUG UUU GAC UAA GCC AGA AUA UCC AGA UAA GGU AGC UGG AGA GAG CAG CGA CUU ACA UCC CCG GUA GAU ACG AAC AGG ACC CCU GCC AUG CAG UGA CCU UUC GUA GCC GCC AGU UCU UGA CCU CUA AGC AGC GUC AGG AUC CGU G-3′ (SEQ ID NO: 1).

To prepare 5′-HS-PEGₙ-GMP-RNA (where n=2 or 4), 5′-HS-PEGₙ-GMP (18b or 18c) was added into the transcription reaction with a ratio of 5′-HS-PEGₙ-GMP to GTP of 1:1, 4:1, 8:1 or 16:1. A 20 μl aliquot of 0.5 MEDTA (pH7.4) was added to dissolve the white precipitate before adding formamide-loading dye. The RNA transcript was purified as described above.

To prepare 5′-HS-G-RNA, 5′-GSMP-RNA was synthesized by runoff transcription in the presence of GSMP with a ratio of GSMP:GTP:ATP:CTP:UTP=8:1:1:1:1 mM. The 5′-GSMP-RNA was dephosphorylated by Calf Intestinal alkaline phosphatase (New England Biolabs) in NEBuffer 3 (50 mM Tris-HCl, 10 mM MgCl₂, 100 mM NaCl, 1 mM dithiothreitol, pH 7.9) at 37° C. for 3 hours to generate 5′-HS-G-RNA. The reaction was stopped by the addition of 10 μl of 200 mM EGTA and incubation at 65° C. for 10 min. The 5′-HS-G-RNA was recovered and resuspended as described above.

Gel shift assays were performed to demonstrate that the 5′-thiol modified RNA was able to conjugate with biological molecules. To test the conjugation of thiol-reactive agents with 5′-HS-G-RNA and 5′-HS-PEG,-RNA, two substrates were used, biotin and maleimidite.

For conjugation with maleimide-activated horseradish peroxidase (HP), a 1.0 μl aliquot of 5′-GTP-RNA or 5′-HS-PEG,-GMP-RNA was incubated with 10 μg of HRP in maleimide conjugation buffer (100 mM sodium phosphate, 5 mM EDTA, pH 7.6) at room temperature for one hour. The HRP-conjugated RNA was then resolved by electrophoresis through an 7.5 M urea/8% polyacrylamide gel. Detection of the HRP-maleimide-RNA conjugate was based on the electrophoretic mobility change of the conjugated RNA which obviated the need to assay for HRP's enzymatic activity. The mobility of KRP labeled RNA will be slower than unmodified RNA on 7.5 M urea/8% polyacrylamide gel.

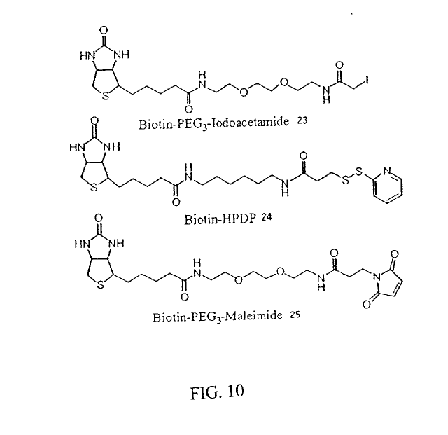

For conjugation with biotin molecules, the thiol-labeled RNAs, 5′-GTP-RNA, 5′-HS-G-RNA, and 5′-HS-PEG.-RNA, were incubated with three different biotin molecules, Biotin-PEG₃-iodoacetamide (23), Biotin-HPDP (24), and Biotin-PEG₃-Maleimide (25) (shown in FIG. 10), in 10 mM HEPES (pH 7.8), 300 mM NaCl, and 1 mM EDTA at room temperature for 2 hr. The reaction mixtures were extracted with phenol/chloroform/ isoamyl alcohol (25:24:1) (pH 6.7) once and chloroform once, and precipitated with ethanol. The RNA pellets were resuspended in 20 μl of pure water and stored at −20° C. A 2 μl aliquot of each of the biotinylated RNAs was incubated with 15 μg of streptavidin in the binding buffer (20 mM HEPES, pH 7.4, 5.0 mM EDTA, and 1.0 M NaCl) at room temperature for 20 min prior to mixing with 0.25 volumes of formamide loading buffer (90% forrnamide; 0.01% bromophenol blue and 0.025% xylene cyanol). The biotinylated RNA products were resolved by electrophoresis through 7.5 M urea polyacrylamide gels. The biotinylated RNA can complex with streptavidin and the mobility of the 5′-biotin-RNA::streptavidin complex through the gel will be retarded relative to unbiotinylated RNA. The fraction of product formation relative to total RNA at each lane was quantitated with a Molecular Dynamics PhosphorImager.

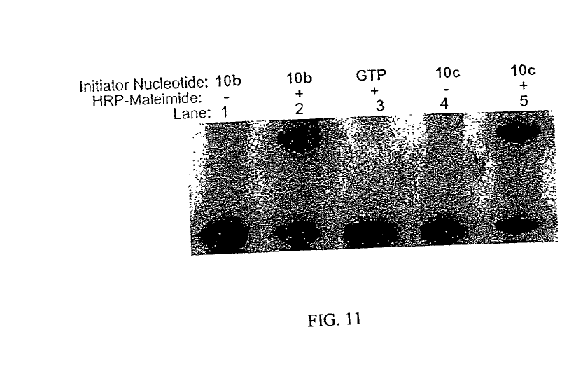

The results from the conjugation of 5′-HS-PEGₙ-GMP-RNA with maleimide-activated Horseradish peroxidase (HRP, MW 40 kD) are shown in FIG. 11. HRP, is one of the most common enzymes used for immunoassay detection systems. Ordinarily the enzyme is detected because it can, under appropriate conditions, form soluble color responses, color precipitates, or generate the chemical emission of light. One commercially available version of horseradish peroxidase contains a thiol-reactive maleimide group enabling the HRP to be introduced efficiently into the 5′-end of the thiol-modified RNA. The change in mass may be detected by an electrophoretic mobility change, thus obviating the need for the bioassay based on HRP's enzymatic activity.

The results of conjugating 5′-HS-PEGₙ-GMP-RNA with the maleimide-activated HRP are demonstrated in FIG. 11. The 5′-HS-PEGₙ-GMP-RNA was incubated with maleimide-activated HRP and detected as an RNA band-shift (lanes 2 and 5), which is the 5′-HRP-S-PEGₙ-GMP-RNA. The overall yield of 5′-HRP-S-PEGₙ-GMP-RNA is 55% with 5′-HS-PEG₄-GMP and 61% with 5′-HS-PEG₂-GMP. Neither 5′-HS-PEG₂-GMP-RNA nor 5′-HS-PEG₄-GMP-RNA demonstrated a retarded band in the absence of the maleimide-activated HRP treatment (lanes 1 and 4). Furthermore, the 5′-GTP-capped-RNA served as a negative control (lane 3). When 5′-GTP-RNA was treated with the maleimide activated HRP, no retarded band was detected. The results suggest that the HRP protein was linked to the 5′-terminal thiol of RNA, not to other functional groups present in RNA. These data suggest that 5′-HS-PEG₂-GMP is a better substrate than 5′-HS-PEG₄-GMP, although both can serve as effective initiators for T7 RNA polymerase. The major advantage of the di-and tetra-ethylene glycol derivatives are that they provide flexible spacers between the RNA and the thiol group, and this flexibility may be important for some bioconjugation applications and immobilized binding studies.

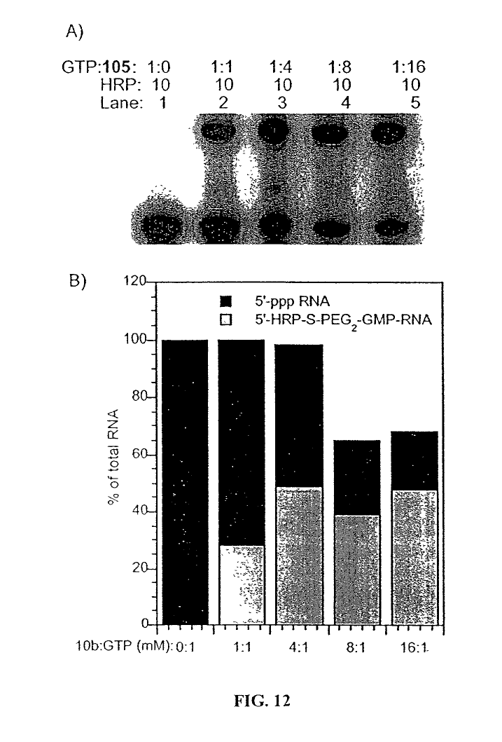

The efficiency of incorporation of 5′-HS-PEG₂-GMP (18b) during in vitro transcription reactions performed with varying molar ratios of GTP to 5′-HS-PEG₂-GMP was examined and the results shown in FIG. 12. The molar ratio of GTP to 5′-HS-PEG₂-GMP (18b) was adjusted by maintaining a consistent concentration of 1 mM GTP while varying the concentration of 5′-HS-PEG₂-GMP (18b) between transcription reactions to produce thiol-containing RNAs.

The thiol-containing RNAs generated by the transcription reactions were conjugated to maleimide-activated HRP during a subsequent incubation step. Assuming that the thiol-maleimide reaction was quantitative, resolution of the 5′-HRP-S-PEG₂-GMP-RNA from the unconjugated RNA allowed the determination of the percent of RNA transcripts that successfully used 5′-HS-PEG₂-GMP (18b) as the initiator nucleotide in lieu of GTP. No 5′-HRP-RNA was formed when 5′-HS-PEG₂-GMP (18b) was absent from the transcription reaction (FIG. 12A, lane 1) confirming that the conjugation of the maleimide-activated HRP with the RNA was dependent upon the use of the thiol-containing initiator nucleotide.

The efficiency of incorporation of 5′-HS-PEG₂-GMP (18b) may be disserned in terms of both relative and absolute yields (i.e. what fraction of the total transcripts were initiated with 5′-HS-PEG₂-GMP (18b), and how many moles of transcripts were produced). This was a necessary distinction since the absolute yield from the transcription reactions decreased at the highest concentrations of 5′-HS-PEG₂-GMP (18b) tested (FIG. 12B). When the ratio of GTP: 5′-HS-PEG₂-GMP (18b) was 1:1, approximately 28% of the nascent transcripts were initiated with 5′-HS-PEG₂-GMP (18b). The percent of transcripts initiated with di(ethylene glycol) monotosylate (10b) increased to 51%, 60%, and 72% as the GTP: 5′-HS-PEG₂-GMP (18b) ratio was varied from 1 mM : 4 mM, 1 mM: 8 mM, and 1 mM: 16 mM, respectively.

FIG. 12B shows that the fraction of 5′-HRP-S-PEG₂-GMP-RNA increased significantly over this interval but the absolute yield of 5′-WRP-S-PEG₂-GMP-RNA remained relatively constant as the absolute total transcription yield (including GTP-initiated transcripts) decreased. When the concentration of 5′-HS-PEG₂-GMP (18b) reached 8 mM, it appeared to slightly inhibit transcription by T7 RNA polymerase. Normalizing the absolute yield of total RNA to 100% when the ratio of GTP to 5′-HS-PEG₂-GMP was 1 mM: 0 mM, the yield decreased to 98% for 1 mM: 4 mM, 65% for 1 mM:8 mM, and 68% for 1 mM: 16 mM transcription reactions.

These results show that 5′-HS-PEG₂-GMP (18b) nucleotide behaves more similar to the effect reported for AMP and GMP addition, specifically a concentration-dependent decrease in yields from transcription reactions utilizing T7 RNA polymerase.

The polyethylene glycols (PEGs) were chosen as linkers because the flexibility that they provide, and because they reduce steric hindrance effects. PEG-containing GMP nucleotides are incorporated less efficiently as initiator nucleotides as the length of the PEG linker increases (Seelig et aL (1999) Bioconjugate Chem. 10, 371-378), therefore, the competing demands of linker flexibility with incorporation efficiency was balanced. The results from FIG. 12 show that an acceptable balance has been found; the initiator nucleotide 5′-HS-PEG₂-GMP (18b), containing two PEG subunits, decreased the absolute total transcription yield when present at a GTP: 5′-HS-PEG₂-GMP (18b) ratio at 1 mM:8-16 mM but without significantly lowering the absolute yield of the desired 5′-HS-PEG₂-GMP (18b)-capped-RNA.

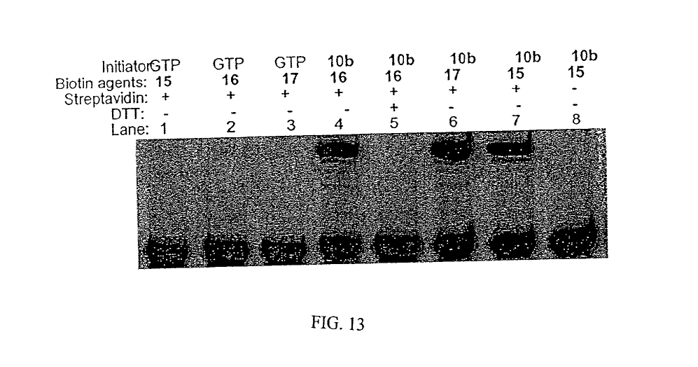

The results from the conjugation of 5′-HS-PEGₙ-GMP-RNA with biotin are shown in FIG. 13. Gel-shift assays were performed using 5′-HS-PEG₂-GMP-RNA conjugation with three different biotinylated thiol-reactive molecules, biotin-PEG₃-Iodoacetamide (23), biotin-HPDP (24), and biotin-PEG₃-Maleimide (25), the structures of which are shown in FIG. 10. The biotin-PEG₃-Iodoacetamide (23), biotin-HPDP (24), and biotin-PEG₃-Maleimide (25) were obtained from Molecular Biosciences, Boulder, Colo.

The 5′thiol-modified RNA bind with the biotinylated molecules, which in turn bind to streptavidin. Streptavidin::RNA complexes are retarded in a gel-shift assay. The streptavidin gel-shift data is presented in FIG. 13. When 5′-HS-PEG₂-GMP-RNA was reacted with biotin-PEG₃-Iodoacetamide (23), biotin-HPDP (24), and biotin-PEG₃-Maleimide (25), the thiol-modified RNA molecules were biotinylated and detected as band shifts in the presence of streptavidin, representing the streptavidin::RNA complexes (FIG. 13, lanes 4, 6, and 7). No retarded band was detected without streptavidin (lane 8).

When 5′-GTP-capped-RNA was treated with biotin-PEG₃-Iodoacetamide (23), biotin-HPDP (24), and biotin-PEG₃-Maleimide (25), no biotinylated RNA was detected in the presence of streptavidin (lanes 1, 2, and 3, respectively). The retarded band disappeared after treatment with DTT (lane 5), which reduced the product of the thiol-disulfide exchange reaction between 5′-HS-RNA and Biotin-HPDP (24). The overall fraction of biotinylated RNA was 39% after reaction with Biotin-HPDP (24), 45% with biotin-PEG₃-Maleimide (25), and 23% with biotin-PEG₃-Iodoacetamide (23) for 5′-HS-PEG₂-GMP (lanes 4, 6, and 7, respectively).

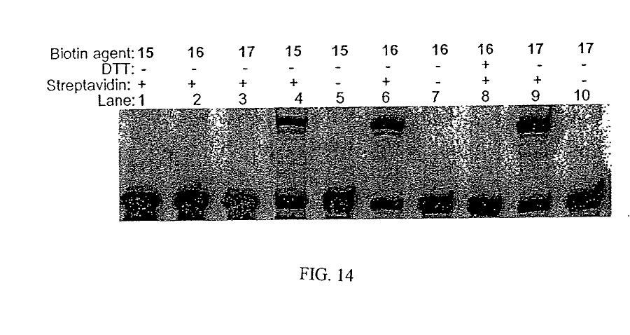

Thiol-reactive biotin conjugation with 5′-HS-G-RNA was also examined, and the results shown in FIG. 14. The bridging phosphorothioate 5′-GSMP-RNA was dephosphorylated by alkaline phosphatase to generate 5′-HS-G-RNA (i.e. RNA containing a 5′ thiol instead of a 5′-hydroxyl group). The streptavidin gel-shift results of 5′-HS-G-RNA following reaction with biotin-PEG₃-Iodoacetamide (23), biotin-EPDP (24), and biotin-PEG₃-Maleimide (25) are shown in FIG. 14. When 5′-HS-RNA was reacted with biotin-PEG₃-Iodoacetamide (23), biotin-HPDP (24), and biotin-PEG₃-Maleimide (25), the thiol-modified RNA molecules were biotinylated and detected as band-shifts in the presence of streptavidin (lanes 4, 6, and 9, respectively); no retarded band was detected without streptavidin (lanes 5, 7, and 10).

No retarded band was detected when 5′-GTP-capped-RNA was treated with biotin-PEG₃-Iodoacetamide (23), biotin-HPDP (24), and biotin-PEG₃-Maleimide (25) (lanes 1, 2, and 3, respectively). The retarded band disappeared after treatment with DTT (lane 8), which reduced the product of the thiol-disulfide exchange reaction between 5′-HS-RNA and Biotin-HPDP (24). These results suggest that the biotin group was transferred to the terminal thiol of the 5′-HS-RNA, not to other nucleophilic groups of RNA. The overall yield (three steps) of biotinylated RNA is 57% with biotin-PEG₃-Iodoacetamide (23), and 60% with biotin-HPDP (24) for GSMP (lane 6 and 9, respectively). The experiments demonstrated that GSMP (22) can serve as a better initiator nucleotide for transcription by T7 RNA polymerase than 5′-HS-PEG₂-GMP (18b) and 5′-HS-PEG₄-GMP (18c) for the purpose of introducing a sulfhydryl group at the 5′-end of RNA.

The thiol-modified RNA molecules of the invention can be used for a number of applications, for example, in the production of RNA bioarray chips. Thiol-modified RNA can be covalently linked to glass or silicon surface, or a polymer sheet via thiol chemical reactions typically used to generate as biochips for RNA, DNA or proteomic arrays (See e.g., U.S. Pat. No. 6,248,521, incorporated herein by reference). The glass surface or polymer sheet can be treated with any one of the haloacetamides, maleimides, benzylic halides or bromomethylketones to attached the thiol-modified RNA to form a chemically stable bond with the surface (e.g., a covalent bond with the surface).

The thiol-modified RNA molecules can be used to bind a number of biological molecules, for example, proteins, peptides, enzymes, carbohydrates, nucleotides, oligonucleotides, DNA, and detectable labels such as fluorophores, biotin, and dyes. The thiol-modified RNA molecules can also be used to bind DNA containing thiol reactive functional group (e.g., haloacetamides, maleimides, benzylic halides or bromomethylketones) to examine nucleic acid-nucleic acid interactions.

All materials can be obtained from commercial sources (Aldrich, Sigma, ACROS, Fisher, USB and VWR) and used without additional purification, unless otherwise noted. Preferably, the solvents are distilled before use. (2-cyanoethyl-N, N-diisopropyl)chlorophosphoramidite was purchased from Peninsula Laboratories. Biotin-PEG₃-iodoacetamide, Biotin-BPDP, and Biotin-PEG₃-Maleimide were purchased from Pierce and Molecular Biosicences. Maleimide-activated horseradish peroxidase was purchased from Pierce, α-³²P-ATP from NEN Lab, NTPs and Taq DNA polymerase from New England Biolabs. All other materials were obtained from Aldrich, Sigma, and Acros, and used without additional purification unless otherwise noted. All solvents were distilled before use. Preferably, dichloromethane, acetonitrile and pyridine should be dried by refluxing with calcium hydride. ¹H, ¹³C, and ³¹P NMR spectra were obtained on Varian 300 and 400 spectrometers. Corresponding operating frequencies were as follows: 299.95/400.14 MHz (¹H), 75.43/100.61 MHz (¹³C), 121.42/161.98 MHz (³¹P). Internal references used are TMS for ¹H and ¹³C, and 85% H₃PO₄ for ³¹p. Mass spectra were obtained on a Finnigan LCQ^{DUO} spectrometer and high resolution MS (FAB) spectra on JMS-700 MStation mass spectrometer.

It should be evident to one of ordinary skill in the art to recognize, or be able to ascertain using no more than routine experimentation, many equivalents to the specific embodiments of the invention described herein. Such equivalents are intended to be encompassed by the following claims. All references cited herein are incorporated by reference in their entirety.

What is claimed is:

1. A method of modifying the 5′-terminus of an RNA molecule, comprising:
obtaining a nucleoside;
reacting the nucleoside with a chemical effective to yield a thiol group onto its 5′-terminus;
isolating the nucleoside comprising 5′-terminus thiol molecule; and
subjecting the nucleoside comprising 5′-terminus thiol molecule to an RNA polymerase molecule, dsDNA and NTPs under conditions suitable for an RNA polymerization reaction.

2. The method of claim 1, wherein the nucleoside is selected from the group consisting of guanosine, uridine, cytidine and adenosine.

3. The method of claim 1, wherein the nucleoside is guanosine.

4. The method of claim 1, wherein the dsDNA comprises at least 50 nucleotide bases per strand of the dsDNA.

5. The method of claim 1, wherein the modified RNA molecule is 5′-GSMP-RNA.

6. The method of claim 4, wherein the 5′-GSMP-RNA is dephophorylated to 5′-HS-G-RNA.

7. The method of claim 1, wherein the modified RNA molecule is 5′-HS-PEG₂-GMP-RNA.

8. The method of claim 1, wherein the modified RNA molecule is 5′-HS-PEG₄-GMP-RNA.


SEQUENCE LISTING (1 sequence)

SEQ ID NO: 1 - RNA, 193 nt, Artificial Sequence
  Synthetic sequence generated by PCR from pC25 DNA template by in vitro transcription with T7 RNA polymerase
gggagagaccugccauucacgcuggauaaaacuucacagccauacguuguguuugacuaagccagaauauccagauaagguagcuggagagagcagcgacuuacauccccgguagauacgaacaggaccccugccaugcagugaccuuucguagccgccaguucuugaccucuaagcagcgucaggauccgug